(19) United States
(12) Patent Application Publication
(10) Pub. No.: US 2003/0171413 A1
(43) Pub. Date: Sep. 11, 2003

(54) Bicyclic heteroaromatic derivatives for the treatment of immune and inflammatory disorders

(76) Inventors: David Alan Owen, Cambridge (GB); Alan Findlay Haughan, Cambridge (GB); Duncan Batty, Haverhill (GB); Alistair George Draffan, Melbourne (AU); Hannah Jayne Kendall, Huntingdon (GB); Sarah Louise Mellor, Royston (GB)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/344,619

(22) PCT Filed: Aug. 16, 2001
(86) PCT No.: PCT/GB01/03721

(51) Int. Cl. C07D 333/66; C07D 333/72

(52) U.S. Cl. 514/367; 514/394; 514/375; 514/443; 514/469; 548/152; 548/217; 548/304.4; 549/55; 549/396

(57) ABSTRACT

Compounds of formula (1) are described, wherein q is zero or the integer 1, 2 or 3; R which when present may be attached to any available carbon or nitrogen atom of the bicyclic heteroaromatic ring of formula (1) is an atom or group -L³(Alk³)_{w}L⁴(R⁸)ᵤ; X is an O atom or a S(O)ₘ atom or group in which m is zero or the integer 1 or 2 or an NR group; Y is a N atom or a CR^{1a} group in which R^{1a} is a group R or a group R¹; R¹ which may he on any available carbon atom of the bicyclic heteroaromatic ring of formula (1) is a hydrogen atom or a group -Alk¹L¹CyAlk²L²D; provided that at least one but not both of R¹ and R^{1a} is the group -Alk¹L¹CyAlk²L²D. The compounds are potent inhibitors of the interaction between CCR-3 and it chemokine ligands and are of use in the prophylaxis and treatment of immune or inflammatory disorders in which inhibition of this interaction can have a beneficial effect.

This invention relates to a series of bicyclic heteroaromatic derivatives, to compositions containing them, to processes for their preparation, and to their use in medicine.

Over the last few years it has become increasingly clear that chemokines (chemotactic cytokines) play a key role in the recruitment and activation of a variety of cell types in inflammatory processes, for example recruitment of eosinophils in the tissue eosinophilia that is a feature of a number of pathological conditions including asthma, rhinitis, eczema and parasitic infections [Schwarz, M. K. and Wells, T. N. C., Curr. Opin. Chem. Biol., 1999, 3, 407-17; Bousquet, J. et al, N. Eng. J. Med., 1990, 323,1033-39; Kay, A. B. and Corrigan, C. J., Br. Med. Bull., 1992, 48, 51-64].

Chemokines are released by a wide variety of cells to attract and activate, among other cell types, macrophages, T and B lymphocytes, eosinophils, basophils and neutrophils [Luster, New Eng. J. Med., 1998, 338, 436-45; Rollins, Blood, 1997, 90, 909-28]. To date almost 40 human chemokines have been well characterised [Schwarz, M. K., ibid; Wells, T. N. C. et al, Trends Pharmacol Sci, 1998, 19, 376-380] and they have been classified into two major classes, CXC and CC, depending on whether the first two cysteines in the amino acid sequence are separated by a single amino acid (CXC) or are adjacent (CC). Members of two additional classes, C chemokines (lymphotactin-1 and lymphotactin-2) and a CX3C chemokine (fractalkine) have also been identified. It was initially thought that CXC chemokines, such as IL-8 (a neutrophil attractant), were associated with acute inflamnmation whilst CC chemokines were associated with chronic inflammatory diseases such as asthma, arthritis and atherosclerosis. However it is now known that members of both classes are involved in both chronic and acute inflammation.

In general the CXC chemokines, such as interleukin-8 (IL-8), neutrophil-activating protein-2 (NAP-2) and melanoma growth stimulatory activating protein (MGSA) are chemotactic primarily for neutrophils and T lymphocytes, whereas CC chemokines such as RANTES (regulation-upon-activation, normal T expressed and secreted), MIP-1α, MIP-1β, the monocyte chemotactic proteins (MCP-1, MCP-2, MCP-3, MCP-4, MCP-5) and the eotaxins (-1, -2 and -3) are chemotactic for macrophages, T lymphocytes, eosinophils, dendritic cells and basophils.

The chemokines bind to specific cell-surface receptors. Seventeen mammalian receptors have been reported to date [Schwarz, M. K. ibid], all of which are seven-transmembrane-spanning G-protein coupled receptors. The ligand binding characteristics of these receptors have been identified, for example the ligands for CCR-1 are RANTES, MIP-1α and MCP-3 whilst those for CCR-2 are MCP-1, 2, 3, 4 and 5.

Chemokines and their receptors have been implicated as important mediators of inflammatory, infectious, and immunoregulatory diseases, as well as autoimmune pathologies such as rheumatoid arthritis and atherosclerosis. For example, in asthma eosinophil accumulation and activation in response to the chemokines RANTES, eotaxin and MCP-3 is associated with damage to bronchial epitheluim and airway hyperresponsiveness to mediators of bronchoconstriction. Of these three chemokines eotaxin alone is selectively chemotactic for eosinophils [Griffith-Johnson, D. A. et al, Biochem. Biophys. Res. Commun., 1993, 197, 1167; Jose, J. P. et al, Biochem. Biophys. Res. Commun.,1994, 207, 788]. Specific eosinophil accumulation was observed at the site of administration of eotaxin via either the intradermal, intraperitoneal or aerosol inhalation route [Griffith-Johnson, ibid; Jose, P. J. et al, J. Exp. Med., 1994, 179, 881-7; Rothenberg, M. E. et al, J. Exp. Med., 1995, 181, 1211; Ponath, P. D., J. Clin. Invest., 1996, 97, 604-12].

Current therapies for eosinophil-related disorders such as bronchial asthma include glucocorticoids (dexamethasone, methprednisolone and hydrocortisone) [Schleimer, R. P. et al, Am. Rev. Respir. Dis., 1990, 141, 559]. The glucocorticoids are believed to inhibit IL-5 and IL-3 mediated eosinophil survival in these diseases. It is known, however, that prolonged use of glucocorticoids can lead to undesirable side effects such as glaucoma, osteoporosis and growth retardation. There is thus a need for alternate means of treating eosinophil mediated disorders.

It has become clear that the CCR-3 chemokine receptor plays a pivotal role in the recruitment of eosinophils to sites of allergic inflammation and in subsequently activating these cells in response to RANTES, eotaxin, MCP-3 and MCP-4 [Ponath, P. D. et al, J. Exp. Med., 1996, 183, 2437-48]. These chemokine ligands for CCR-3 induce a rapid increase in intracellular calcium concentration, increased expression of cellular adhesion molecules, cellular degranulation and the promotion of eosinophil migration. The release of lipid mediators, cytotoxic proteins, oxygen metabolites and cytokines by eosinophils upon activation all have the potential to produce a pathophysiological response. The CCR-3 receptor is expressed on the surface of eosinophils, T-cells (subtype Th-2) and to a lesser extent basophils and mast cells and is the only known chemokine receptor for eotaxin. It has been shown that pretreatment with an anti-CCR-3 monoclonal antibody completely inhibits eosinophil chemotaxis to eotaxin, RANTES and MCP-3 [Heath, H. et al, J. Clin. Invest., 1997, 99, 178-84]. The restricted expression of CCR-3 on eosinophils and T-cells may be responsible for the selective recruitment of eosinophils and Th-2 T-cells in allergic inflammation.

Mammalian cytomegaloviruses, herpesviruses and poxviruses have been shown to express, in infected cells, proteins with the binding properties of chemokine receptors (Wells and Schwartz, Curr. Opin. Biotech., 1997, 8, 741-48). Human CC chemokines (e.g. RANTES and MCP-3) can cause rapid mobilization of calcium via these virally encoded receptors, the expression of which may allow for infection by permitting subversion of the normal immune system surveillance and response to infection. Additionally human chemokine receptors (e.g. CXCR4, CCR2, CCR3, CCR5 and CCR8) can act as co-receptors for the infection of mammalian cells by microbes such as the human immunodeficiency virus (HIV).

Accordingly there is a great need for agents that modulate the ability of chemokines to bind to chemokine receptors, particularly agents that block the ability of RANTES, eotaxin, MCP-3 and MCP-4 to bind to CCR-3, thus preventing the recruitment of eosinophils and so providing a method of treatment for eosinophil-mediated inflammatory diseases. We have found a class of bicyclic heteroaromatic derivatives that are potent inhibitors of the interaction between CCR-3 and its chemokine ligands. The compounds are thus of use in medicine, for example in the prophylaxis and treatment of Immune or inflammatory disorders as described hereinafter.

Thus according to one aspect of the invention we provide a compound of formula (1):

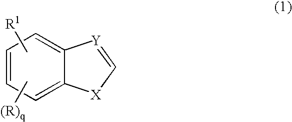

wherein:

q is zero or the integer 1, 2 or 3;

R which when present may be attached to any available carbon or nitrogen atom of the bicyclic heteroaromatic ring of formula (1) is an atom or group -L³(Alk³)_{w}L⁴(R⁸)ᵤ in which L³ and L⁴ which may be the same or different is each a covalent bond or a linker atom or group, w is zero or the integer 1, u is the integer 1, 2 or 3, Alk³ is an optionally substituted aliphatic or heteroaliphatic chain and R⁸ is a hydrogen or halogen atom or a group selected from alkyl, —OR⁹ [where R⁹ is a hydrogen atom or an optionally substituted alkyl group], —SR⁹, —NR⁹R¹⁰, [where R¹⁰ is as just defined for R⁹ and may be the same or different]. —NO₂, —CN, —CO₂R⁹, —OCO₂R⁹, —CONR⁹R¹⁰, —OCONR⁹R¹⁰, —CSNR⁹R¹⁰, —COR⁹, —OCOR⁹, —N(R⁹)COR¹⁰, —N(R⁹)CSR¹⁰, —SO₂N(R⁹)(R¹⁰) , —N(R⁹)SO₂R¹⁰, —N(R⁹)CON(R¹⁰)(R¹¹), [where R¹¹ is a hydrogen atom or an optionally substituted alkyl group], —N(R⁹)CSN(R¹⁰)(R¹¹), —N(R⁹)SO₂N(R¹⁰)(R¹¹) or an optionally substituted cycloaliphatic, heterocycloaliphatic, aromatic or heteroaromatic group provided that when w is zero and each of L³ and L⁴ is a covalent bond then u is the integer 1; or

when q is the integer 2 or 3 and two R groups are attached to adjacent carbon atoms of the heteroaromatic ring of formula (1) these may be joined together with the heteroaromatic ring carbon atoms to form an optionally substituted cycloaliphatic, heterocycloaliphatic aromatic or heteroaromatic group;

X is an O atom or a S(O)ₘ atom or group in which m is zero or the integer 1 or 2 or an NR group;

Y is a N atom or a CR^{1a} group in which R^{1a} is a group R or a group R¹;

R¹ which may be on any available carbon atom of the bicyclic heteroaromatic ring of formula (1) is a hydrogen atom or a group -Alk¹L¹CyAlk²L²D in which Alk¹ is an optionally substituted C₁₋₃alkyl group, Alk² is an optionally substituted aliphatic or cycloaliphatic group, L¹ is a —CON(R⁵)—, —N(R⁵)CO—, —SO₂N(R⁵)— or —N(R⁵)SO₂— group in which R⁵ is a hydrogen atom or an optionally substituted aliphatic, heteroaliphatic, cycloaliphatic, polycycloaliphatic, heterocycloaliphatic, heteropolycycloaliphatic, aromatic, or heteroaromatic group, L² is a covalent bond or an —O— atom or —S(O)ₙ— atom or group in which n is zero or the integer 1 or 2 or —N(R⁷)— group in which R⁷ is a hydrogen atom or an optionally substituted aliphatic, heteroaliphatic, cycloaliphatic, polycycloaliphatic, heterocycloaliphatic, heteropolycycloaliphatic, aromatic, or heteroaromatic group, D is an optionally substituted aliphatic, heteroaliphatic, cycloaliphatic, polycycloaliphatic, heterocycloaliphatic, heteropolycycloaliphatic, aromatic, or heteroaromatic group and Cy is an optionally substituted heterocycloaliphatic. ring of formula (A) or (B):

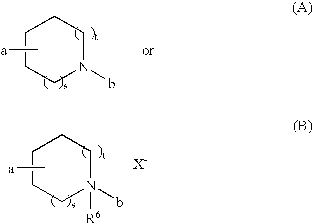

in which

a indicates the point of attachment of any available ring carbon in the ring Cy to the group L¹, b indicates the point of attachment to Alk², s and t which may be the same or different is each zero or the integer 1 or 2, provided that s+t is the integer 1, 2, 3 or 4, R⁶ is an optionally substituted alkyl group and X is a pharmaceutically acceptable counterion;

provided that at least one but not both of R¹ and R^{1a} is the group -Alk¹L¹CyAlk²L²D;

and the salts, solvates, hydrates, N-oxides thereof.

It will be appreciated that certain compounds of formula (1) may exist as geometric isomers (E or Z isomers) The compounds may also have one or more chiral centres, and exist as enantiomers or diastereomers. The invention is to be understood to extend to all such geometric isomers, enantiomers, diastereomers and mixtures thereof, including racemates. Formula (1) and the formulae hereinafter are intended to represent all individual isomers and mixtures thereof, unless stated or shown otherwise. In addition, compounds of formula (1) may exist as tautomers, for example keto (CH₂C═O)— enol (CH═CHOH) tautomers. Formula (1) and the formulae hereinafter are intended to represent all individual tautomers and mixtures thereof, unless stated otherwise.

In the compounds of the invention as represented by formula (1) and the more detailed description hereinafter certain of the general terms used in relation to substituents are to be understood to include the following atoms or groups unless specified otherwise.

Thus as used herein the term “alkyl”, whether present as a group or part of a group includes straight or branched C₁₋₁₀alkyl groups, for example C₁₋₆alkyl groups such as methyl, ethyl, n-propyl, i-propyl, n-butyl, i-butyl, s-butyl or t-butyl groups and C₃₋₁₀cycloalkyl groups, for example C₃₋₆cycloalkyl groups such as cyclopropyl, cyclobutyl, cyclopentyl and cyclohexyl groups. Similarly, the terms “alkenyl” or “alkynyl” are intended to mean straight or branched C₂₋₁₀alkenyl or C₂₋₁₀alkynyl groups such as C₂₋₆alkenyl or C₂₋₆alkynyl groups such as —CHCH₂, —CHCHCH₃, —CH₂CHCHCH₃, —CCH, —CH₂CCH and —CH₂CCCH₃ groups. Optional substituents present on those groups include those optional substituents mentioned hereinafter in relation to optionally substituted aliphatic groups.

The term “halogen atom” is intended to include fluorine, chlorine, bromine or iodine atoms.

The term “haloalkyl” is intended to include the alkyl groups just mentioned substituted by one, two or three of the halogen atoms just described. Particular examples of such groups include —CF₃, —CCl₃, —CHF₂, —CHCl₂, —CH₂F, and —CH₂Cl groups.

The term “alkoxy” as used herein is intended to include straight or branched C₁₋₁₀alkoxy for example C₁₋₆alkoxy such as methoxy, ethoxy, n-propoxy,i-propoxy and t-butoxy. “Haloalkoxy” as used herein includes any of those alkoxy groups substituted by one, two or three halogen atoms as described above. Particular examples include —OCF₃, —OCCl₃, —OCHF₂, —OCHCl₂, —OCH₂F and —OCH₂Cl groups.

As used herein the term “alkylthio” is intended to include straight or branched C₁₋₁₀alkylthio, e.g. C₁₋₆alkylthio such as methylthio or ethylthio groups.

The term “aliphatic group” is intended to include optionally substituted straight or branched C₁₋₁₀alkyl, e.g. C₁₋₆alkyl, C₂₋₁₀alkenyl e.g. C₂₋₆alkenyl or C₂₋₁₀alkynyl e.g. C₂₋₆alkynyl groups.

The term “heteroaliphatic group” is intended to include the optionally substituted aliphatic groups just described but with each group additionally containing one, two, three or four heteroatoms or heteroatom-containing groups. Particular heteroatoms or groups include atoms or groups L⁵ where L⁵ is a linker atom or group. Each L⁵ atom or group may interrupt the aliphatic group, or may be positioned at its terminal carbon atom to connect the group to an adjoining atom or group. Particular examples of suitable L⁵ atoms or groups include —O— or —S— atoms or —C(O)—, —C(O)O—, —C(S)—, —S(O), —S(O)₂—, —N(R¹⁷)— [where R¹⁷ is a hydrogen atom or an alkyl group], —N(R¹⁷)N(R¹⁷)—, —N(R¹⁷)O—, —CON(R¹⁷)—, —OC(O)N(R¹⁷)—, —CSN(R¹⁷)—, —N(R¹⁷)CO—, —N(R¹⁷)C(O)O—, —N(R¹⁷)CS—, —S(O)₂N(R¹⁷)—, —N(R¹⁷)S(O)₂—, —N(R¹⁷)CON(R¹⁷)—, —N(R¹⁷)CSN(R¹⁷)—, or —N(R¹⁷)SO₂N(R¹⁷)— groups. Where the linker group contains two R¹⁷ substituents, these may be the same or different.

Particular examples of aliphatic groups include optionally substituted —CH₃, CH₂CH₃, —CH(CH₃)₂, —(CH₂)₂CH₃, —(CH₂)₃CH₃, —CH(CH₃)CH₂CH₃, —CH₂CH(CH₃)₂, —C(CH₃)₃, —(CH₂)₄CH₃, —(CH₂)₅CH₃, —CHCH₂, —CHCHCH₃, —CH₂CHCH₂, —CHCHCH₂CH₃, —CH₂CHCHCH₃, —(CH₂)₂CHCH₂, —CCH, —CCCH₃, —CH₂CCH, —CCCH₂CH₃, —CH₂CCCH₃, or —(CH₂)₂CCH groups. Where appropriate each of said groups may be optionally interrupted by one, two, three or more atoms and/or groups L⁵ to form an optionally substituted heteroaliphatic group. Particular examples include optionally substituted -L⁵CH₃, —CH₂L⁵CH₃, -L⁵CH₂CH₃, -L⁵CH₂CHCH₂, -L⁵CH₂CCH, —CH₂L⁵CH₂CH₃, -L⁵CH₂L⁵CH₃, —(CH₂)₂L⁵CH₃, -L⁵(CH₂)₂CH₃ and —(CH₂)₂L⁵CH₂CH₃ groups.

The optional substituents which may be present on aliphatic or heteroaliphatic groups include one, two, three or more substituents where each substituent may be the same or different and is selected from halogen atoms, or alkoxy, hydroxy (—OH), thiol (—SH), alkylthio, amino (—NH₂); substituted amino, optionally substituted C₆₋₁₂arylamino, —CN, —CO₂H, —CO₂R¹² (where R¹² is an alkyl group), —SO₃H, —SOR¹², —SO₂R¹², —SO₃R¹², —OCO₂R¹², —C(O)H, —C(O)R¹², —OC(O)R¹², —C(S)R¹², —C(O)N(R¹³)(R¹⁴) (where R¹³ and R¹⁴, which may be the same or different is each a hydrogen atom or an alkyl group), —OC(O)N(R¹³)(R¹⁴), —N(R¹³)C(O)R¹⁴, —CSN(R¹³)(R¹⁴), —N(R¹³)C(S)(R¹⁴), —SO₂N(R¹³)(R¹⁴), —N(R¹³)SO₂R¹⁴, —N(R¹³)C(O)N(R¹⁴)(R¹⁵) (where R¹⁵ is a hydrogen atom or an alkyl group), —N(R¹³)C(S)N(R¹⁴)(R¹⁵), —N(R¹³)SO₂N(R¹⁴)(R¹⁵), or an optionally substituted cycloaliphatic, heterocycloaliphatic, aromatic or heteroaromatic group. Substituted amino groups include —NHR¹² and —N(R¹²)(R¹³) groups. Optionally substituted cycloaliphatic, heterocycloaliphatic, aromatic and heteroaromatic groups include those groups described hereinafter.

The term optionally substituted aliphatic or heteroaliphatic chain is intended to include those optionally substituted aliphatic and heteroaliphatic groups as just described where a terminal hydrogen atom is replaced by a covalent bond to give a divalent chain.

The term “cycloaliphatic group” is intended to include optionally substituted C₃₋₁₀ cycloaliphatic groups. Particular examples include optionally substituted C₃₋₁₀cycloalkyl, e.g. C₃₋₈cycloalkyl or C₃₋₁₀cycloalkenyl, e.g C₃₋₈cycloalkenyl groups.

The term “heterocycloaliphatic group” is intended to include optionally substituted C₃₋₁₀heterocycloaliphatic groups. Particular examples include optionally substituted C₃₋₁₀heterocycloalkyl, e.g. C₃₋₇heterocycloalkyl, or C₃₋₁₀heterocycloalkenyl, e.g. C₃₋₇hetercycloalkenyl groups, each of said groups containing one, two, three or four heteroatoms or heteroatom-containing groups L⁵ as just defined.

The term “polycycloaliphatic group” is intended to include optionally substitued C₇₋₁₀bi- or tricycloalkyl or C₇₋₁₀bi- or tricycloalkenyl groups. The term “heteropolycycloaliphatic group” is intended to include the optionally substituted polycycloaliphatic groups just described, but with each group additionally containing one, two, three or four L⁵ atoms or groups.

Particular examples of cycloaliphatic, polycycloaliphatic, heterocycloaliphatic and heteropolycycloaliphatic groups include optionally substituted cyclopropyl, cyclobutyl, cyclopentyl, cyclohexyl, cycloheptyl, cyclooctyl, cyclobutenyl, cyclopentenyl, cyclopentenyl, cyclohexenyl, cycloheptenyl, cyclooctenyl, adamantyl, norbornyl, norbornenyl, tetrahydrofuranyl, tetrahydropyranyl, pyrroline, e.g. 2- or 3-pyrrolinyl, pyrrolidinyl, pyrrolidinone, oxazolidinyl, oxazolidinone, dioxolanyl, e.g. 1,3-dioxolanyl, imidazolinyl, e.g. 2-imidazolinyl, imidazolidinyl, pyrazolinyl, e.g. 2-pyrazolinyl, pyrazolidinyl, thiazolinyl, thiazolidinyl, pyranyl, e.g. 2- or 4-pyranyl, piperidinyl, piperidinone, 1,4-dioxanyl, morpholinyl, morpholinone, 1,4-dithianyl, thiomorpholinyl, piperazinyl, 1,3,5-trithianyl, oxazinyl, e.g. 2H-1,3-, 6H-1,3-, 6H-1,2-, 2H-1,2- or 4H-1,4-oxazinyl, 1,2,5-oxathiazinyl, isoxazinyl, e.g. o- or p-isoxazinyl, oxathiazinyl, e.g. 1,2,5 or 1,2,6-oxathiazinyl, or 1,3,5,-oxadiazinyl or succinimidyl groups.

Cycloaliphatic and polycycloaliphatic groups may be linked to the remainder of the compound of formula (1) by any available ring carbon atom. Heterocycloaliphatic and heteropolycycloaliphatic groups may be linked to the remainder of the compound of formula (1) by any available ring carbon or, where available, ring nitrogen atom.

The optional substituents which may be present on the cycloaliphatic, polycycloaliphatic, heterocycloaliphatic or heteropolycycloaliphatic groups include one, two, three or more optionally substituted alkyl groups and/or optional substituents as described above in relation to aliphatic or heteroaliphatic groups.

The terms “aromatic group” and “aryl group” are intended to include for example optionally substituted monocyclic or bicyclic fused ring C₆₋₁₂ aromatic groups, such as phenyl, 1- or 2-naphthyl, 1- or 2-tetrahydronaphthyl, indanyl or indenyl groups. Each of these aromatic groups may be optionally substituted by one, two, three or more R¹⁹ atoms or groups as defined below.

The terms “heteroaromatic group” and “heteroaryl group” are intended to include for example optionally substituted C₁₋₉heteroaromatic groups containing for example one, two, three or four heteroatoms selected from oxygen, sulphur or nitrogen atoms. In general, the heteroaromatic groups may be for example monocyclic or bicyclic fused ring heteroaromatic groups. Monocyclic heteroaromatic groups include for example five- or six-membered heteroaromatic groups containing one, two, three or four heteroatoms selected from oxygen, sulphur or nitrogen atoms. Bicyclic heteroaromatic groups include for example eight- to thirteen-membered fused-ring heteroaromatic groups containing one, two or more heteroatoms selected from oxygen, sulphur or nitrogen atoms.

The aromatic and heteroaromatic groups may be attached to the remainder of the compound of formula (1) by any carbon or hetero e.g. nitrogen atom as appropriate.

Particular examples of heteroaromatic groups of these types include pyrrolyl, furyl, thienyl, imidazolyl, N—C₁₋₆alkylimidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, 1,3,4-thiadiazole, pyridyl, pyrimidinyl, pyridazinyl, pyrazinyl, 1,3,5-triazinyl, 1,2,4-triazinyl, 1,2,3-triazinyl, benzofuryl, [2,3-dihydro]benzofuryl, benzothienyl, benzotriazolyl, indolyl, indolinyl, isoindolyl, indazolinyl, benzimidazolyl, imidazo[1,2-a]pyridyl, benzothiazolyl, benzoxazolyl, benzisoxazolyl, benzopyranyl, [3,4-dihydro]benzopyranyl, quinazolinyl, quinoxalinyl, naphthyridinyl, pyrido[3,4-b]pyridyl, pyrido[3,2-b]pyridyl, pyrido[4,3-b]-pyridyl, quinolinyl, isoquinolinyl, phthalazinyl, tetrazolyl, 5,6,7,8-tetrahydroquinolinyl or 5,6,7,8-tetrahydroisoquinolinyl.

Optional substituents which may be present on the aromatic or heteroaromatic groups include one, two, three or more substituents, each selected from an atom or group R¹⁹ in which R¹⁹ is —R^{19a} or -Alk⁴(R^{19a})_{f}, where R^{19a} is a halogen atom, or an amino (—NH₂), substituted amino, nitro, cyano, amidino, hydroxyl (—OH), substituted hydroxyl, formyl, carboxyl (—CO₂H), esterified carboxyl, thiol (—SH), substituted thiol, —COR²⁰ [where R²⁰ is an -Alk⁴(R^{19a})_{f}, aryl or heteroaryl group], —CSR²⁰, —SO₃H, —SOR²⁰, —SO₂R²⁰, —SO₃R²⁰, —SO₂NH₂, —SO₂NHR²⁰, SO₂N(R²⁰)₂, —CONH₂, —CSNH₂, —CONHR²⁰, —CSNHR²⁰, —CON(R²⁰)₂, —CSN(R²⁰)₂, —N(R²¹)SO₂R²⁰, [where R²¹ is a hydrogen atom or an alkyl group] —N(SO₂R²⁰)₂, —N(R²¹)SO₂NH₂, —N(R²¹)SO₂NHR²⁰, —N(R²¹)SO₂N(R²⁰)₂, —N(R²¹)COR²⁰, —N(R²¹)CONH₂, —N(R²¹)CONHR²⁰, —N(R²¹)CON(R²⁰)₂, —N(R²¹)CSNH₂, —N(R²¹)CSNHR²⁰, —N(R²¹)CSN(R²⁰)₂, —N(R²¹)CSR²⁰, —N(R²¹)C(O)OR²⁰,—SO₂NHet¹ [where —NHet¹ is an optionally substituted C₅₋₇cyclicamino group optionally containing one or more other —O— or —S— atoms or —N(R²¹)—, —C(O)— or —C(S)— groups], —CONHet¹, —CSNHet¹, —N(R²¹)SO₂NHet¹, —N(R²¹)CONHet¹, —N(R²¹)CSNHet¹, —SO₂N(R²¹)Het² [where Het² is an optionally substituted monocyclic C₅₋₇carbocyclic group optionally containing one or more —O— or —S— atoms or —N(R²¹)—, —C(O)— or —C(S)— groups], -Het², —CON(R²¹)Het², —CSN(R²¹)Het², —N(R²¹)CON(R²¹)Het², —N(R²¹)CSN(R²¹)Het², aryl or heteroaryl group; Alk⁴ is a straight or branched C₁₋₆alkylene, C₂₋₆alkenylene or C₂₋₆alkynylene chain, optionally interrupted by one, two or three —O— or —S— atoms or —S(O)_{g}—[where g is an integer 1 or 2] or —N(R²¹)— groups; and f is zero or an integer 1, 2 or 3. It will be appreciated that when two R²⁰ or R²¹ groups are present in one of the above substituents, the R²⁰ or R²¹ groups may be the same or different.

When in the group -Alk⁴(R^{19a})_{f} f is an integer 1, 2 or 3, it is to be understood that the substituent or substituents R^{19a} may be present on any suitable carbon atom in -Alk⁴. Where more than one R^{19a} substituent is present these may be the same or different and may be present on the same or different atom in -Alk⁴. Clearly, when f is zero and no substituent R^{19a} is present the alkylene, alkenylene or alkynylene chain represented by Alk⁴ then Alk⁴ becomes an alkyl, alkenyl or alkynyl group.

When R^{19a} is a substituted amino group it may be for example a group —NHR²⁰ [where R²⁰ is as defined above] or a group —N(R²⁰)₂ wherein each R²⁰ group is the same or different.

When R^{19a} is a substituted hydroxyl or substituted thiol group it may be for example a group —OR²⁰ or a —SR²⁰ or —SC(═NH)NH₂ group respectively.

Esterified carboxyl groups represented by the group R^{19a} include groups of formula —CO₂Alk⁵ wherein Alk⁵ is an alkyl group; a C₆₋₁₂arylC₁₋₈alkyl group such as an optionally substituted benzyl, phenylethyl, phenylpropyl, 1-naphthylmethyl or 2-naphthylmethyl group; an aryl group; a C₆₋₁₂aryloxyC₁₋₈alkyl group such as an optionally substituted phenyloxymethyl, phenyloxyethyl, 1-naphthyloxymethyl, or 2-naphthyloxymethyl group; an optionally substituted C₁₋₈alkanoyloxyC₁₋₈alkyl group, such as a pivaloyloxymethyl, propionyloxyethyl or propionyloxypropyl group; or a C₆₋₁₂aroyloxyC₁₋₈alkyl group such as an optionally substituted benzoyloxyethyl or benzoyloxypropyl group. Optional substituents present on the Alk⁵ group include R^{19a} substituents described above.

When Alk⁴ is present in or as a substituent it may be for example a methylene, ethylene, n-propylene, i-propylene, n-butylene, i-butylene, s-butylene, t-butylene, ethenylene, 2-propenylene, 2-butenylene, 3-butenylene, ethynylene, 2-propynylene, 2-butynylene or 3-butynylene chain, optionally interrupted by one, two, or three —O— or —S—, atoms or —S(O)—, —S(O)₂— or —N(R²¹)— groups.

When —NHet¹ or —Het² forms part of a substituent R¹⁹ each may be for example an optionally substituted 2- or 3-pyrrolinyl, pyrrolidinyl, pyrazolinyl, pyrazolidinyl, piperazinyl, imidazolinyl, imidazolidinyl, morpholinyl, thiomorpholinyl, piperidinyl, oxazolidinyl or thiazolidinyl group. Additionally Het² may represent for example, an optionally substituted cyclopentyl or cyclohexyl group. Optional substituents which may be present on —NHet¹ or —Het² include those substituents described above in relation to aromatic groups.

Particularly useful atoms or groups represented by R¹⁹ include fluorine, chlorine, bromine or iodine atoms, or C₁₋₆alkyl, e.g. methyl, ethyl, n-propyl, i-propyl, n-butyl or t-butyl, optionally substituted phenyl, pyridyl, pyrimidinyl, pyrrolyl, furyl, thiazolyl, thienyl, morpholinyl, thiomorpholinyl, piperazinyl, pyrroiidinyl or piperidinyl, C₁₋₆hydroxyalkyl, e.g. hydroxymethyl or hydroxyethyl, carboxyC,₁₋₆alkyl, e.g. carboxyethyl, C₁₋₆alkylthio e.g. methylthio or ethylthio, carboxyC₁₋₆alkylthio, e.g. carboxymethylthio, 2-carboxyethylthio or 3-carboxypropylthio, C₁₋₆alkoxy, e.g. methoxy or ethoxy, hydroxyC₁₋₆alkoxy, e.g. 2-hydroxyethoxy, optionally substituted phenoxy, pyridyloxy, thiazolyoxy, phenylthio or pyridylthio, C₅₋₇cycloalkoxy, e.g. cyclopentyloxy, haloC₁₋₆alkyl, e.g. trifluoromethyl, haloC₁₋₆alkoxy, e.g. trifluoromethoxy, C₁₋₆alkylamino, e.g. methylamino or ethylamino, amino (—NH₂), aminoC₁₋₆alkyl, e.g. aminomethyl or aminoethyl, C₁₋₆dialkylamino, e.g. dimethylamino or diethylamino, aminoC₁₋₆alkylamino e.g. aminoethylamino, Het¹NC₁₋₆alkylamino e.g. morpholinopropylamino, C₁₋₆alkylaminoC₁₋₆alkyl, e.g. ethylaminoethyl, C₁₋₆dialkylaminoC₁₋₆alkyl, e.g. diethylaminoethyl, aminoC₁₋₆alkoxy, e.g. aminoethoxy, C₁₋₆alkylaminoC₁₋₆alkoxy, e.g. methylaminoethoxy, C₁₋₆dialkylaminoC₁₋₆alkoxy, e.g. dimethylaminoethoxy, diethylaminoethoxy, diisopropylaminoethoxy, or dimethylaminopropoxy, hydroxyC₁₋₆alkylamino e.g. hydroxyethylamino, imido, such as phthalimido or naphthalimido, e.g. 1,8-naphthalimido, nitro, cyano, amidino, hydroxyl (—OH), formyl [HC(O)—], carboxyl (—CO₂H), —CO₂Alk⁵ [where Alk⁵ is as defined above], C₁₋₆alkanoyl e.g. acetyl, optionally substituted benzoyl, thiol (—SH), thioC₁₋₆alkyl, e.g. thiomethyl or thioethyl, —SC(═NH)NH₂, sulphonyl (—SO₃H), —SO₃R²⁰, C₁₋₆alkylsulphinyl e.g. methylsulphinyl, C₁₋₆alkylsulphonyl, e.g. methylsulphonyl, aminosulphonyl (—SO₂NH₂), C₁₋₆alkylaminosulphonyl, e.g. methylamino-sulphonyl or ethylaminosulphonyl, C₁₋₆dialkylaminosulphonyl, e.g. dimethyl-aminosulphonyl or diethylaminosulphonyl, optionally substituted phenylamino-sulphonyl, carboxamido (—CONH₂), C₁₋₆alkylaminocarbonyl, e.g. methylamino-carbonyl or ethylaminocarbonyl, C₁₋₆dialkylaminocarbonyl, e.g. dimethyl-aminocarbonyl or diethylaminocarbonyl, aminoC₁₋₆alkylaminocarbonyl, e.g. aminoethylaminocarbonyl, C₁₋₆dialkylaminoC₁₋₆alkylaminocarbonyl, e.g. diethylaminoethylaminocarbonyl, aminocarbonylamino, C₁₋₆alkylamino-carbonylamino, e.g. methylaminocarbonylamino or ethylaminocarbonylamino, C₁₋₆dialkylaminocarbonylamino, e.g. dimethylaminocarbonylamino or diethyl-aminocarbonylamino, C₁₋₆alkylaminocabonylC₁₋₆alkylamino, e.g. methylamino-carbonylmethylamino, aminothiocarbonylamino, C₁₋₆alkylaminothiocarbonyl-amino, e.g. methylaminothiocarbonylamino or ethylaminothiocarbonylamino, C₁₋₆dialkylaminothiocarbonylamino, e.g. dimethylaminothiocarbonylamino or diethylaminothiocarbonylamino, C₁₋₆alkylaminothiocarbonylC₁₋₆alkylamino, e.g. ethylaminothiocarbonylmethylamino, —CONHC(=NH)NH₂, C₁₋₆alkyl-sulphonylamino, e.g. methylsulphonylamino or ethylsulphonylamino, C₁₋₆dialkylsulphonylamino, e.g. dimethylsulphonylamino or diethylsulphonyl-amino, optionally substituted phenylsulphonylamino, aminosulphonylamino (—NHSO₂NH₂), C₁₋₆alkylaminosulphonylamino, e.g. methylaminosulphonylamino or ethylaminosulphonylamino, C₁₋₆dialkylaminosulphonylamino, e.g. dimethylaminosulphonylamino or diethylaminosulphonylamino, optionally substituted morpholinesulphonylamino or morpholinesulphonylC₁₋₆alkylamino, optionally substituted phenylaminosulphonylamino, C₁₋₆alkanoylamino, e.g. acetylamino, aminoC₁₋₆alkanoylamino e.g. aminoacetylamino, C₁₋₆dialkyl-amino C₁₋₆alkanoylamino, e.g. dimethylaminoacetylamino, C₁₋₆alkanoyl-amino C₁₋₆alkyl, e.g. acetylaminomethyl, C₁₋₆alkanoylaminoC₁₋₆alkylamino, e.g. acetamidoethylamino, C₁₋₆alkoxycarbonylamino, e.g. methoxycarbonylamino, ethoxycarbonylamino or t-butoxycarbonylamino or optionally substituted benzyloxy, benzylamino, pyridylmethoxy, thiazolylmethoxy, benzyloxy-carbonylamino, benzyloxycarbonylaminoC₁₋₆alkyl e.g. benzyloxycarbonyl-aminoethyl, thiobenzyl, pyridylmethylthio or thiazolylmethylthio groups.

Where desired, two R¹⁹ substituents may be linked together to form a cyclic group such as a cyclic ether, e.g. a C₁₋₆alkylenedioxy group such as methylenedioxy or ethylenedioxy, or two R¹⁹ substituents may form a cycloimidyl group, for example to form an imidyl group in for example phthalimidyl, or naphthalimidyl such as 1,8-naphthalimidyl.

It will be appreciated that where two or more R¹⁹ substituents are present, these need not necessarily be the same atoms and/or groups. In general, the substituent(s) may be present at any available ring position in the aromatic or heteroaromatic group.

Examples of C₁₋₃alkyl groups represented by the group Alk¹ in the group R¹ include —CH₂—, —CH₂CH₂—, —CH₂CH₂CH₂—, —CH(CH₃)CH₂— and —CH₂CH(CH₃)— groups. Optional substituents which may be present on any carbon atom of the group Alk¹ include one, two, three or more optional substituents selected from optionally substituted aliphatic, heteroaliphatic, cycloaliphatic, polycycloaliphatic, heterocycloaliphatic, heteropolycycloaliphatic, aromatic, or heteroaromatic groups as herein defined. When two or more optional substituents are present on carbon atoms of the group Alk¹ these may be joined together with the carbon atoms of Alk¹ to which they are attached to form a cyclic structure. Particular examples include joining two aliphatic or heteroaliphatic chains to form, together with the carbons to which they are attached, a cycloaliphatic or heterocycloaliphatic ring.

Aliphatic and cycloaliphatic groups represented by Alk² in the group R¹ include those aliphatic and cycloaliphatic groups as herein defined. Optional substituents that may be present on Alk² aliphatic and cycloaliphatic groups include halogen atoms. Thus for example, Alk² may be a straight or branched C¹⁻¹⁰ alkyl, C₂₋₁₀ alkenyl or C₂₋₁₀ alkynyl group as defined herein, optionally substituted by one, two, three or more halogen atoms.

In compounds of the invention D may for example be an optionally substituted aliphatic, heteroaliphatic, cycloaliphatic, polycycloaliphatic, heterocycloaliphatic, heteropolycycloaliphatic, 1- or 2-naphthyl, 1- or 2-tetrahydronaphthyl, indanyl, indenyl, or heteroaromatic group as defined herein for compounds of formula (1).

It will be understood that such Alk² aliphatic and cycloaliphatic groups are divalent groups that may be joined to Cy and L² in the group R¹ via any available carbon atom or atoms.

Examples of substituents represented by R¹ when present in compounds of the invention include groups -Alk¹L¹CyAlk²L²D and -Alk¹L¹CyAlk²D. Particular examples of such substituents include —CH₂L¹CyCH₂L²D, —CH₂L¹CyCH₂D, —CH₂CH₂L¹CyCH₂L²D, —CH₂CH₂L¹CyCH₂D, —CH₂CH(CH₃)L¹CyCH₂L²D, —CH₂L¹CyCH₂CH₂L²D, —CH₂L¹CyCH₂CH₂D, or —CH₂L¹CyCH(CH₃)CH₂L²D. More particular examples of such substituents include —CH₂CON(R⁵)CyCH₂L²D, —CH₂N(R⁵)COCyCH₂L²D, —CH₂SO₂N(R⁵)CyCH₂L²D and —CH₂N(R⁵)SO₂CyCH₂L²D. In compounds of these types L² may be for example, an —O— atom or —S(O)ₙ— atom or group in which n is zero or the integer 1 or 2 or —N(R⁷)— group.

Optional substituents which may be present on any available carbon of the ring Cy include one, two or three substituents, R^{6A}, where R^{6A} is a halogen atom or an alkyl group. Thus for example Cy may be substituted by a halogen atom or a straight or branched C₁₋₁₀alkyl group as defined herein.

Examples of heterocycloaliphatic rings represented by Cy in compounds of the invention include:

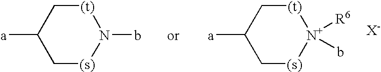

where s is the integer 1, t is zero or the integer 2 and a, b X³¹ and R⁶ are as described earlier, or

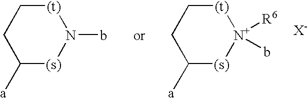

where s is the integer 1 or 2, t is zero or the integer 1 or 2 and a, b X³¹ and R⁶ are as described earlier.

Linker atoms or groups L³ and L⁴, when present in the group R in compounds of the invention may be any of the linker atoms or groups as previously defined for L⁵. Each linker atom or group may be the same or different.

When the groups R⁹ and R¹⁰ or R¹⁰ and R¹¹ are both present in the group R as alkyl groups these groups may be joined together with the N atom to which they are attached to form a heterocyclic ring. Such heterocyclic rings may be optionally interrupted by a further heteroatom selected from —O—, —S— or —N(R⁹)—. Particular examples of such heterocyclic rings include piperidinyl, morpholinyl, thiomorpholinyl, pyrrolidinyl, imidazolidinyl and piperazinyl rings.

Example of the substituents represented by R when present in compounds of the invention include atoms or groups -L³Alk³L⁴R⁸, -L³Alk³R⁸, -L³R⁸, —R⁸, -Alk³R⁸ and -Alk³(R⁸)ᵤ wherein L³, Alk³, L⁴, R⁸ and u are as defined above. Particular examples of such substituents include -L³CH₂L⁴R⁸, -L³CH(CH₃)L⁴R⁸, -L³(CH₂)₂L⁴R⁸, -L³CH₂R⁸, -L³CH(CH₃)R⁸, -L³(CH₂)₂R⁸, —CH₂R⁸, —CH(CH₃)R⁸, —(CH₂)₂R⁸ and —R⁸ groups.

Particularly useful atoms or groups represented by R in compounds of the invention include for example by one, two, three or more halogen atoms, e.g. fluorine, chlorine, bromine or iodine atoms, and/or C₁₋₆alkyl, e.g. methyl, ethyl, n-propyl, i-propyl, n-buty( or t-butyl, optionally substituted C₃₋₈cycloalkyl, e.g. optionally substituted cyclopropyl, cyclobutyl, cyclopentyl or cyclohexyl, optionally substituted C₃₋₇heterocycloalkyl, e.g. optionally substituted pyrrolidinyl, piperidinyl, imidazolidinyl, morpholinyl, or piperazinyl, C₁₋₆hydroxyalkyl, e.g. hydroxymethyl, hydroxyethyl or —C(OH)(CF₃)₂, carboxyC₁₋₃alkyl, e.g. carboxyethyl, C₁₋₆alkylthio e.g. methylthio or ethylthio, carboxyC₁₋₆alkylthio, e.g. carboxymethylthio, 2-carboxyethylthio or 3-carboxypropylthio, C₁₋₆alkoxy, e.g. methoxy or ethoxy, hydroxyC₁₋₆alkoxy, e.g. 2-hydroxyethoxy, haloC₁₋₆alkyl, e.g. —CF₃, —CHF₂, —CH₂F, haloC₁₋₆alkoxy, e.g. —OCF₃, —OCHF₂; —OCH₂F, C₁₋₆alkylamino, e.g. methylamino or ethylamino, amino (—NH₂), aminoC₁₋₆alkyl e.g. aminomethyl or aminoethyl, C₁₋₆dialkylamino, e.g. dimethylamino or diethylamino, C₁₋₆alkylaminoC₁₋₆alkyl, e.g. ethylaminoethyl, C₁₋₆ dialkylaminoC₁₋₆alkyl, e.g. diethylaminoethyl, aminoC₁₋₆alkoxy, e.g. aminoethoxy, C₁₋₆alkylaminoC₁₋₆alkoxy, e.g. methylaminoethoxy, C₁₋₆dialkylaminoC₁₋₆alkoxy, e.g. dimethylaminoethoxy, diethylaminoethoxy, diisopropylaminoethoxy, or dimethylamino-propoxy, nitro, cyano, amidino, hydroxyl (—OH), formyl [HC(O)—], carboxyl (—CO₂H), —CO₂Alk⁵ [where Alk⁵ is as previously defined], C₁₋₆ alkanoyl e.g. acetyl, thiol (—SH), thioC₁₋₆alkyl, e.g. thiomethyl or thioethyl, sulphonyl (—SO₃H), —SO₃Alk⁵, C₁₋₆alkylsulphinyl, C₁₋₆alkylsulphonyl, e.g. methylsulphonyl, amino-sulphonyl (—SO₂NH₂), C₁₋₆alkylaminosulphonyl, e.g. methylaminosulphonyl or ethylaminosu(phonyl, C₁₋₆dialkylaminosulphonyl, e.g. dimethylaminosulphonyl or diethylamino-sulphonyl, phenylaminosulphonyl, carboxamido (—CONH₂), C₁₋₆alkyl-aminocarbonyl, e.g. methylaminocarbonyl or ethylaminocarbonyl, C₁₋₆dialkylaminocarbonyl, e.g. dimethylaminocarbonyl or diethylaminocarbonyl, aminoC₁₋₆alkylaminocarbonyl, e.g. aminoethylaminocarbonyl, C₁₋₆dialkylamino-C₁₋₆alkylaminocarbonyl, e.g. diethylaminoethylaminocarbonyl, aminocarbonylamino, C₁₋₆alkylaminocarbonyl-amino, e.g. methylaminocarbonylamino or ethyl-aminocarbonylamino, C₁₋₆dialkylaminocarbonylamino, e.g. dimethylaminocarbonylamino or diethyl-aminocarbonylamino, C₁₋₆alkylaminocabonylC₁₋₆alkyl-amino, e.g. methylamino-carbonylmethylamino, aminothiocarbonylamino, C₁₋₆alkylaminothiocarbonyl-amino, e.g. methylaminothiocarbonylamino or ethylaminothiocarbonylamino, C₁₋₆dialkylaminothiocarbonylamino, e.g. dimethyl-aminothiocarbonylamino or diethylaminothiocarbonylamino, C₁₋₆alkylaminothio-carbonylC₁₋₆alkylamino, e.g. ethylaminothiocarbonylmethylamino, C₁₋₆alkylsulphonylamino, e.g. methyl-sulphonylamino or ethylsulphonylamino, C₁₋₆dialkylsulphonylamino, e.g. dimethylsulphonylamino or diethylsulphonylamino, aminosulphonylamino (—NHSO₂NH₂), C₁₋₆alkylaminosulphonylamino, e.g. methylaminosulphonylamino or ethylaminosulphonylamino, C₁₋₆dialkylaminosulphonylamino, e.g. dimethyl-aminosulphonylamino or diethylaminosulphonylamino, C₁₋₆alkanoylamino, e.g. acetylamino, aminoC₁₋₆alkanoylamino e.g. aminoacetylamino, C₁₋₆dialkylamino-C₁₋₆alkanoylamino, e.g. dimethylaminoacetylamino, C₁₋₆alkanoylaminoC₁₋₆alkyl, e.g. acetylaminomethyl, C₁₋₆alkanoylaminoC₁₋₆alkylamino, e.g. acetamido-ethylamino, C₁₋₆alkoxycarbonylamino, e.g. methoxycarbonylamino, ethoxy-carbonylamino or t-butoxycarbonylamino groups, optionally substituted C₆₋₁₂aryl e.g. optionally substituted phenyl, optionally substituted C₁₋₉ heteroaryl, e.g optionally substituted pyridyl, pyrimidinyl, thiophenyl or furyl, optionally substituted C₁₋₆alkylC₆₋₁₂aryl, e.g optionally substituted benzyl or phenylethyl, optionally substituted C₁₋₆alkylC₁₋₉heteroaryl, e.g. optionally substituted pyridylmethyl, furanylmethyl or thiophenylmethyl, C₁₋₆alkoxyC₆₋₁₂aryl, e.g optionally substituted benzyloxy or phenylethoxy, optionally substituted C₁₋₆alkoxyC₁₋₉heteroaryl, e.g. optionally substituted pyridylmethoxy, furanylmethoxy or thiophenylmethoxy groups.

Where desired two R substituents may be linked together to form a cyclic group such as a cyclic ether, e.g. a C₁₋₆alkylenedioxy group such as methylenedioxy or ethylenedioxy. In addition when two R substituents are on adjacent carbon atoms of the heteroaromatic ring of formula (1) they may be joined to form, together with the heteroaromatic ring carbon atoms to which they are joined, a heteroaromatic ring fused optionally substituted cycloaliphatic, heterocycloaliphatic, aromatic or heteroaromatic ring where such rings and optional substituents are as previously defined.

It will be appreciated that where two or more R substituents are present these need not necessarily be the same atoms and/or groups. In general the substituent(s) may be present on any available ring position in the heteroaromatic ring het in compounds of formula (1).

R⁶ when present in compounds of formula (1) as an optionally substituted alkyl group may be any optionally substituted alkyl group as previously defined. Particular examples of such groups include C₁₆alkyl groups and optionally substituted C₆₋₁₂arylC₁₋₆alkyl groups, especially methyl, ethyl and optionally substituted benzyl groups.

A pharmaceutically acceptable counterion means an ion having a charge opposite to that of the substance with which it is associated and that is pharmaceutically acceptable. Representative examples include, but are not limited to, chloride, bromide, iodide, methanesulfonate, p-tolylsulfonate, trifluoroacetate, acetate and the like as described in Remington's Pharmaceutical Sciences, 17^{th} ed., Mack Publishing Company, Easton, Pa., 1985.

The presence of certain substituents in the compounds of formula (1) may enable salts of the compounds to be formed. Suitable salts include pharmaceutically acceptable salts, for example acid addition salts derived from inorganic or organic acids, and salts derived from inorganic and organic bases.

Acid addition salts include hydrochlorides, hydrobromides, hydroiodides, alkylsulphonates, e.g. methanesulphonates, ethanesulphonates, or isothionates, arylsulphonates, e.g. p-toluenesulphonates, besylates or napsylates, phosphates, sulphates, hydrogen sulphates, acetates, trifluoroacetates, propionates, citrates, maleates, fumarates, malonates, succinates, lactates, oxalates, tartrates and benzoates.

Salts derived from inorganic or organic bases include alkali metal salts such as sodium or potassium salts, alkaline earth metal salts such as magnesium or calcium salts, and organic amine salts such as morpholine, piperidine, dimethylamine or diethylamine salts.

Particularly useful salts of compounds according to the invention include pharmaceutically acceptable salts, especially acid addition pharmaceutically acceptable salts.

X in compounds of the invention is preferably an O or S atom or a NR² group. Especially useful NR² groups include NH, NCH₃ and NCH₂Ph where Ph is an optionally substituted phenyl ring. A most especially preferred X group is an O atom. Thus one group of compounds of the invention has the formula (1a):,

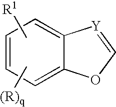

wherein R¹, R, Y and q are as defined for formula (1).

A particularly useful group of compounds according to the invention has the formula (2):

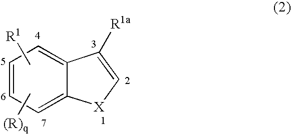

where the numbers 1 to 7 indicate the atom numbering of the heteroaromatic ring according to IUPAC nomenclature and R, q, R¹, R^{1a} and X are as defined for formula (1) or (1a).

and the salts, solvates, hydrates, N-oxides thereof.

In general in compounds of formula (1), (1a) and (2) q is preferably zero or the integer 1 or 2.

Each R atom or group when present in compound of formula (2) may be independently selected from an atom or group -L³(Alk³)_{w}L⁴(R⁸)ᵤ in which L³, Alk³, w, L⁴, R⁸ and u are as previously defined. Particularly useful R substituents when present in compounds of formula (2) include halogen atoms, especially fluorine, chlorine or bromine atoms, or methyl, halomethyl, especially —CF₃ and —CHF₂, methoxy or halomethoxy, especially —OCF₃ or —OCHF₂, methylendioxy, ethylenedioxy, —CN, —CO₂R⁹, especially —CO₂CH₃, —COR⁹, especially —COCH₃, —NO₂, amino (—NH₂), substituted amino (—NR⁹R¹⁰) and —N(R⁹)COR¹⁰, especially —NHCOCH₃ groups.

In one preferred class of compounds of formula (1) and (2) R^{1a} is a group -Alk¹L¹CyAlk²L²D.

In another preferred class of compounds of formula (1) and (2) R¹ is a group -Alk¹L¹CyAlk²L²D and R^{1a} is an atom or group R. In one preferred group of compounds in this class R^{1a} is a hydrogen atom.

In another preferred class of compounds of formula (1), (1a) and (2) R¹ is a group -Alk¹L¹CyAlk²L²D where R¹ is attached to the carbon atom numbered 4.

In another preferred class of compounds of formula (1), (1a) and (2) R¹ is a group -Alk¹L¹CyAlk²L²D where R¹ is attached to the carbon atom numbered 5.

In another preferred class of compounds of formula (1), (1a) and (2) q is the integer 1 and R is attached to the carbon atom numbered 2. In one preferred group of compounds of this class R is a halogen atom, especially a fluorine, chlorine or bromine atom. In another preferred group of compounds of this class R is the group —SR⁹ or —OR⁹. In this group of compounds R⁹ is preferably a —CH₃, —CH₂CH₃ or —CH(CH₃)₂ group. Most especially preferred R atoms or groups in this class of compounds include chlorine atoms and —SCH₃ groups.

In general in compounds of formula (1), (1a) and (2) Alk¹ in the substituent -Alk¹L¹CyAlk²L²D is preferably an optionally substituted —CH₂— or —CH₂CH₂— group. When Alk¹ is substituted it is preferably substituted with an optionally substituted aliphatic group, in particular —CH₃, —CH₂CH₃, —CH(CH₃)₂, —(CH₂)₂CH₃, —CH(CH₃)CH₂CH₃, —CH₂CH(CH₃)₂, or —C(CH₃)₃, especially —CH(CH₃)₂ or —C(CH₃)_{3.}

In general in compounds of formula (1), (1a) and (2) L¹ in the substituent -Alk¹L¹CyAlk²L²D is preferably a —CON(R⁵)— group. R⁵ in this L¹ group is preferably a hydrogen atom or a C₁₋₆alkyl group, especially a —CH₃ or. —CH₂CH₃ group.

In one preferred class of compounds of formula (1), (1a) and (2) R¹ is the group -Alk¹L¹CyAlk²L²D in which L¹ is preferably the group —CON(R⁵)— in which R⁵ is preferably a hydrogen atom or —CH₃ or —CH₂CH₃ group, L² is preferably a covalent bond and Alk² is preferably an optionally substituted C₁₋₆, aliphatic group. Especially useful Alk² groups include optionally substituted —CH₂—, —CH(CH₃)— and —CH₂CH₂— groups.

In another preferred class of compounds of formula (1), (1a) and (2) R¹ is the group -Alk¹L¹CyAlk²L²D in which L¹ is preferably the group —CON(R⁵)— in which R⁵ is preferably a hydrogen atom or —CH₃ or —CH₂CH₃ group, L² is preferably an —O— or —S— atom or —N(R⁷)— group in which R⁷ is preferably a hydrogen atom or —CH₃ or —CH₂CH₃ group and Alk² is preferably an optionally substituted C₁₋₆aliphatic group. A most especially useful L² atom is an —O— atom. Especially useful Alk² groups in this class of compounds include optionally substituted —CH₂—, —CH(CH₃)— and —CH₂CH₂— groups.

In another preferred class of compounds of formula (1), (1a) and (2) s and t in the heterocycloaliphatic ring Cy are each the integer 1.

In another preferred class of compounds of formula (1), (1a) and (2) D is an optionally substituted cycloaliphatic group, particularly an optionally substituted C₃₋₈cycloalkyl or C₃₋₈cycloalkenyl group. Particularly preferred optionally substituted C₃₋₈cycloalkyl groups include cyclopentyl, cyclohexyl, cycloheptyl and cyclooctyl groups. Particularly preferred optionally substituted C₃₋₈cycloalkenyl groups include cyclopentenyl, cyclohexenyl, cycloheptenyl and cyclooctenyl groups.

Compounds according to the invention are potent and selective inhibitors of chemokine binding to the CCR-3 receptor. The ability of the compounds to act in this way may be simply determined by employing tests such as those described in the Examples hereinafter.

The compounds are of use in modulating chemokine mediated cell signalling and in particular are of use in the prophylaxis and treatment of diseases or disorders involving inappropriate migration of cells. The invention extends to such a use and to the use of the compounds of formula (1) for the manufacture of a medicament for treating such diseases and disorders. Particular diseases include inflammatory diseases and immune disorders.

Particular uses to which the compounds of the invention may be put include the treatment or inhibition of asthma, especially bronchial asthma, eczema, conjunctivitis, allergic rhinitis, nasal polyposis, atopic dermatitis, pruritis, psoriasis, chronic obstructive pulmonary disease, adult respiratory distress syndrome, arthritis, Crohn's disease, ulcerative colitis, inflammatory bowel disease, septic shock, endotoxic shock, gram negative sepsis, toxic shock syndrome, stroke, cardiac and renal reperfusion injury, glomerulonephritis, thrombosis, Alzheimer's disease, graft vs host rejection, allograft rejection, HIV infection, rheumatoid arthritis, Acquired Immune Deficiency Syndrome and atherosclerosis.

For the prophylaxis or treatment of disease the compounds according to the invention may be administered as pharmaceutical compositions, and according to a further aspect of the invention we provide a pharmaceutical composition which comprises a compound of formula (1) together with one or more pharmaceutically acceptable carriers, excipients or diluents.

Pharmaceutical compositions according to the invention may take a form suitable for oral, buccal, parenteral, nasal, topical, vaginal or rectal administration, or a form suitable for administration by inhalation or insufflation.

For oral administration, the pharmaceutical compositions may take the form of, for example, tablets, lozenges or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium glycollate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents, emulsifying agents, non-aqueous vehicles and preservatives. The preparations may also contain buffer salts, flavouring, colouring and sweetening agents as appropriate.

Preparations for oral administration may be suitably formulated to give controlled release of the active compound.

For buccal administration the compositions may take the form of tablets or lozenges formulated in conventional manner.

The compounds for formula (1) may be formulated for parenteral administration by injection e.g. by bolus injection or infusion. Formulations for injection may be presented in unit dosage form, e.g. in glass ampoule or multi dose containers, e.g. glass vials. The compositions for injection may take such forms as suspensions, solutions or emulsions in oily or aqueous vehicles, and may contain formulatory agents such as suspending, stabilising, preserving and/or dispersing agents. Alternatively, the active ingredient may be in powder form for constitution with a suitable vehicle, e.g. sterile pyrogen-free water, before use. For particle mediated administration the compounds of formula (1) may be coated on particles such as microscopic gold particles.

In addition to the formulations described above, the compounds of formula (1) may also be formulated as a depot preparation. Such long acting formulations may be administered by implantation or by intramuscular injection.

For nasal administration or administration by inhalation, the compounds for use according to the present invention are conveniently delivered in the form of an aerosol spray presentation for pressurised packs or a nebuliser, with the use of suitable propellant, e.g. dichlorodifluoromethane, trichloro-fluoromethane, dichlorotetrafluoroethane, carbon dioxide or other suitable gas or mixture of gases.

For vaginal or rectal administration the compounds of formula (1) may be formulated as a suppository. These formulations may be prepared by mixing the active ingredient with a suitable non-irritating excipient which is a solid at room temperature but liquid at the body temperature. Such materials include for example cocoa butter and polyethylene glycols.

The compositions may, if desired, be presented in a pack or dispenser device which may contain one or more unit dosage forms containing the active ingredient. The pack or dispensing device may be accompanied by instructions for administration.

The quantity of a compound of the invention required for the prophylaxis or treatment of a particular condition will vary depending on the compound chosen, and the condition of the patient to be treated. In general, however, daily dosages may range from around 100 ng/kg to 100 mg/kg e.g. around 0.01 mg/kg to 40 mg/kg body weight for oral or buccal administration, from around 10 ng/kg to 50 mg/kg body weight for parenteral administration and around 0.05 mg to around 1000 mg e.g. around 0.5 mg to around 1000 mg for nasal administration or administration by inhalation or insufflation.

The compounds of the invention may be prepared by a number of processes as generally described below and more specifically in the Examples hereinafter. Many of the reactions described are well-known standard synthetic methods which may be applied to a variety of compounds and as such can be used not only to generate compounds of the invention, but also where necessary the intermediates thereto.

In the following process description, the symbols Alk¹, h, R³, R, q, Y, X, Cy, R¹⁷, L², Alk², n, D, R^{1a}, Alk³, w, L⁴, R⁸, u, Alk⁵, R⁹ and R²⁰ when used in the formulae depicted are to be understood to represent those groups described above in relation to formula (1) unless otherwise indicated. In the reactions described below, it may be necessary to protect reactive functional groups, for example hydroxy, amino, thio or carboxy groups, where these are desired in the final product, to avoid their unwanted participation in the reactions.

Conventional protecting groups may be used in accordance with standard practice [see, for example, Green, T. W. in “Protective Groups in Organic Synthesis”, John Wiley and Sons, (1999) and the examples herein]. In some instances, deprotection may be the final step in the synthesis of a compound of formula (1) and the processes according to the invention described hereinafter are to be understood to extend to such removal of protecting groups.

Thus according to a further aspect of the invention, a compound of formula (1) in which L¹ is a —CON(R⁵)— group may be obtained by coupling of a compound of formula (3):

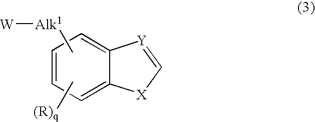

in Which W is a —CO₂H group with a compound of formula HN(R⁵)CyAlk²L²D.

The reaction may be performed in the presence of a base, such as a hydride, e.g. sodium hydride or an amine, e.g. triethylamine or N-methylmorpholine, in a solvent such as a halogenated hydrocarbon, e.g. dichloromethane or carbon tetrachloride or an amide, e.g. dimethylformamide at for example ambient temperature in the presence of a condensing agent, for example a diimide such as 1-(3-dimethylaminopropyl)-3-ethylcarbodimide or N, N′-dicyclohexylcarbodiimide, advantageously in the presence of a catalyst such as a N-hydroxy compound e.g. a N-hydroxytriazole such as 1-hydroxytriazole. Alternatively the acid in compounds of formula (3) may be activated by for example conversion to an acid halide such as an acid chloride by reaction with a halogenating agent such as thionyl chloride or oxalyl chloride in a solvent such as a halogenated hydrocarbon e.g. dichloromethane or converted to a chloroformate, for example ethyl chloroformate, prior to the desired coupling reaction under the conditions just described.

A compound of formula (1) in which L¹ is a —SO₂N(R⁵)— group may be obtained by coupling of a compound of formula (3) in which W is a —SO₂Cl group with a compound of formula HN(R⁵)CyAlk²L²D under the reaction conditions just described for the coupling of acid halides of formula (3).

It will be appreciated that similar reagents and conditions can be used to obtain compounds of formula (1) in which L¹ is a —N(R⁵)CO— or —N(R⁵)SO₂— group using a compound of formula (3) in which W is a —N(R⁵)H group and a compound of formula V-CyAlk²L²D where V is a HO₂C— or ClO₂S— group.

Alternatively compounds of formula (1) may be prepared by reaction of a compound of formula (3A):

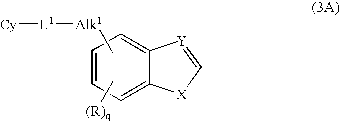

with an Alk^{2a}L²D group, wherein Alk^{2a} is a suitable precursor to Alk², for example Alk^{2a} contains a reactive group, such as a carbonyl or a leaving group e.g. a halogen. This reaction may be achieved using methods known to those skilled in the art. In the case where Alk^{2a} incorporates a carbonyl group, such as a ketone or an aldehyde, this may be reacted with (3A) in the presence of a suitable reducing agent to give a compound of formula (1). Appropriate conditions may include the use of a suitable borohydride as reductant, for example sodium triacetoxyborohydride or sodium cyanoborohydride, in a solvent such as a halogenated hydrocarbon, e.g. dichloromethane, a ketone such as acetone, or an alcohol, e.g. methanol or ethanol, where necessary in the presence of an acid such as acetic acid at around ambient temperature.

Intermediates of formula (3A) may be prepared using standard coupling procedures as described above for the synthesis of compounds of formula (1). For example a compound of formula (3) may be reacted with a HN(R⁵)Cy group using conditions described herein. It will be appreciated that for optimal results reactive sites may be suitably protected prior to reaction and then subsequently removed, using standard techniques.

Intermediates of formula (3), (3A) and any other intermediates required to obtain compounds of formula (1) may be prepared by methods known to those skilled in the art following procedures set forth in references such as Rodd's Chemistry of Carbon Compounds, Volumes 1-15 and Supplementals (Elsevier Science Publishers, 1989), Fieser and Fieser's Reagents for Organic Synthesis, Volumes 1-19 (John Wiley and Sons, 1999), Comprehensive Heterocyclic Chemistry, Ed. Katritzky et al, Volumes 1-8, 1984 and Volumes 1-11, 1994 (Pergamon), Comprehensive Organic Functional Group Transformations, Ed. Katritzky et al, Volumes 1-7, 1995 (Pergamon), Comprehensive Organic Synthesis, Ed. Trost and Flemming, Volumes 1-9, (Pergamon, 1991), Encyclopedia of Reagents for Organic Synthesis Ed. Paquette, Volumes 1-8 (John Wiley and Sons, 1995), Larock's Comprehensive Organic Transformations (VCH Publishers Inc., 1989) and March's Advanced Organic Chemistry (John Wiley and Sons, 1992)

Thus, for example, intermediates of formula HN(R⁵)CyAlk²L²D, may be prepared by reductive alkylation of a compound of formula HN(R⁵)Cy with an Alk^{2a}L²D group, in which Alk^{2a} is a defined herein, using methods known to those skilled in the art. Appropriate conditions may include the use of a suitable borohydride as reductant, for example sodium triacetoxyborohydride or sodium cyanoborohydride, in a solvent such as a halogenated hydrocarbon, e.g. dichloromethane, a ketone such as acetone, or an alcohol, e.g. methanol or ethanol, where necessary in the presence of an acid such as acetic acid at around ambient temperature.

Intermediates of formula (3) in which X and Y are both heteroatoms may be prepared by reaction of intermediates of formula (4):

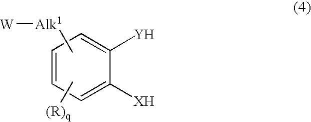

with for example an acylating agent or derivative thereof such as an acid halide of formula RCOHal [where Hal is a halogen atom such as a chlorine atom], a xanthate salt e.g. a salt of ethyl xanthate such as the potassium salt (CH₃CH₂OC(═S)SK), an amidine (RC(=NH)NH2) or a urea (H₂NC(=NH)NHR).

The reaction may be performed optionally in the presence of a base such as a hydride, e.g. sodium hydride or an amine, e.g. triethylamine or N-methylmorpholine, optionally in a solvent such as a halogenated hydrocarbon, e.g. dichloromethane or carbon tetrachloride or an amide, e.g. dimethylformamide or an alcohol such as methanol or ethanol at a temperature from ambient temperature to the reflux temperature.

Intermediates of formula (3) in which Y is the group CR^{1a} and X is the group NH may be prepared from optionally substituted anilines of formula PhNH₂ and (α-halomethylketones of formula HalCH₂COR^{1a} by initial alkylation of the aniline followed by acid catalysed cyclization to give an intermediate of formula (3).

The alkylation may be carried out in the presence of a base such as a carbonate, e.g. caesium or potassium carbonate, an alkoxide, e.g. potassium-butoxide, a hydride, e.g. sodium hydride or an organic amine, e.g. triethylamine or N,N-diisopropylethylamine or a cyclic amine, e.g. N-methylmorpholine or pyridine, in a dipolar aprotic solvent such as an amide, e.g. a substituted amide such as dimethylformamide or an ether, e.g. a cyclic ether such as tetrahydrofuran.

Acid catalyzed cyclization may be performed using an acid such as polyphosphoric acid or a Lewis acid such as aluminium chloride optionally in the presence of a solvent such as a halogenated hydrocarbon, e.g. dichloromethane.

Intermediates of formula (3) in which Y is the group CR^{1a} and X is an O atom may be prepared from an optionally substituted phenol of formula PhOH and a halide of formula R²⁹O₂CCH(Hal)COR^{1a} [where R²⁹ is an alkyl group] by initial alkylation of the phenol followed by acid catalysed cyclization, under the conditions just described, to give an intermediate of formula (3).

Intermediates of formulae (3), HN(R⁵)CyAlk²L²D, HN(R⁵)Cy and OHCAlk²L²D may be further derivatised by one or more standard synthetic methods employing substitution, oxidation, reduction or cleavage reactions. Particular substitution approaches include conventional alkylation, arylation, heteroarylation, acylation, thioacylation, halogenation, sulphonylation, nitration, formylation and coupling procedures. It will be appreciated that these methods may also be used to obtain or modify other compounds of formulae (1) and (2) where appropriate functional groups exist in these compounds.

Intermediates of formula (3) in which R is a group NHR^{a} where R^{a} is an aromatic or heteroaromatic group may be prepared by reaction of an intermediate of formula (3) where R is an NH₂ group with a compound of formula Z³R^{a} [where Z³ is a halogen atom such as a bromine or iodine atom or a trifluoromethanesulfonate group]. The reaction may be carried out in the presence of a metal complex catalyst such as a palladium complex, e.g. dichloro[1,1′-bis(diphenylphosphino)ferrocene]palladium (II), optionally in the presence of an organic base, for example an alkoxide such as sodium t-butoxide, in a solvent such as an ether e.g. a cyclic ether such as tetrahydrofuran, at an elevated temperature e.g. the reflux temperature.

Intermediates of formula (3) in which R is a group -(Alk³)_{w}L⁴(R⁸)ᵤ may be prepared by reaction of an intermediate of formula (3) in which R is a halogen atom such as a bromine or iodine atom with an organometallic reagent HalM(Alk³)_{w}L⁴(R⁸)ᵤ, where M is a metal atom such as a zinc or magnesium atom and Hal is a halogen atom such as a bromine atom. The reaction may be carried out in the presence of a metal catalyst such as a palladium complex, e.g. tetrakis(triphenylphosphine)palladium (0), in a solvent such as an ether, e.g. a cyclic ether such as tetrahydrofuran, at an elevated temperature, e.g. the reflux temperature.

Compounds of the invention and intermediates thereto may be prepared by alkylation, arylation or heteroarylation. For example, compounds containing a -L²H or -L³H group (where L² and L³ is each a linker atom or group) may be treated with an alkylating agent DZ¹ or (R⁸)ᵤL⁴(Alk³)_{w}Z¹ respectively in which Z¹ is a leaving atom or group such as a halogen atom, e.g. a fluorine, bromine, iodine or chlorine atom or a sulphonyloxy group such as an alkylsulphonyloxy, e.g. trifluoromethylsulphonyloxy or arylsulphonyloxy, e.g. p-toluenesulphonyloxy group.

The reaction may be carried out in the presence of a base such as a carbonate, e.g. caesium or potassium carbonate, an alkoxide, e.g. potassium t-butoxide, or a hydride, e.g. sodium hydride, in a dipolar aprotic solvent such as an amide, e.g. a substituted amide such as dimethylformamide or an ether, e.g. a cyclic ether such as tetrahydrofuran.

In another example, compounds containing a -L³H group as defined above may be functionalised by acylation or thioacylation, for example by reaction with one of the alkylating agents just described but in which Z¹ is replaced by a —C(O)Z², C(S)Z², —N(R¹⁷)COZ²or —N(R¹⁷)C(S)Z² group in which Z² is a leaving atom or group as described for Z¹. The reaction may be performed in the presence of a base, such as a hydride, e.g. sodium hydride or an amine, e.g. triethylamine or N-methylmorpholine, in a solvent such as a halogenated hydrocarbon, e.g. dichloromethane or carbon tetrachloride or an amide, e.g. dimethylformamide, at for example ambient temperature. Alternatively, the acylation may be carried out under the same conditions with an acid (for example one of the alkylating agents described above in which Z¹ is replaced by a —CO₂H group) in the presence of a condensing agent, for example a diimide such as 1-(3-dimethylaminopropyl)-3-ethylcarbodiimide or N,N′-dicyclohexylcarbodiimide, or a benzotriazole such as [O-(7-azabenzo-triazol-1-yl)-1,1,3,3-tetramethyluronium]hexafluorophosphate advantageously in the presence of a catalyst such as a N-hydroxy compound e.g. a N-hydroxytriazole such as 1-hydroxybenzotriazole. Alternatively the acid may be reacted with a chloroformate, for example ethylchloroformate, prior to the desired acylation reaction.

In a further example compounds may be obtained by sulphonylation of a compound containing an —OH group by reaction with one of the above alkylating agents but in which Z¹ is replaced by a —S(O)Hal or —SO₂Hal group [in which Hal is a halogen atom such as chlorine atom] in the presence of a base, for example an inorganic base such as sodium hydride in a solvent such as an amide, e.g. a substituted amide such as dimethylformamide at for example ambient temperature.

In another example, compounds containing a -L²H or -L³H group as defined above may be coupled with one of the alkylation agents just described but in which Z¹ is replaced by an —OH group in a solvent such as tetrahydrofuran in the presence of a phosphine, e.g. triphenylphosphine and an activator such as diethyl, diisopropyl- or dimethylazodicarboxylate.

In a further example, ester groups —CO₂R¹² or —CO₂Alk⁵ in the compounds may be converted to the corresponding acid [—CO₂H] by acid- or base-catalysed hydrolysis depending on the nature of the groups R12 or Alk⁵. Acid- or base-catalysed hydrolysis may be achieved for example by treatment with an organic or inorganic acid, e.g. trifluoroacetic acid in an aqueous solvent or a mineral acid such as hydrochloric acid in a solvent such as dioxan or an alkali metal hydroxide, e.g. lithium hydroxide in an aqueous alcohol, e.g. aqueous methanol. Similarly an acid [—CO₂H] may be prepared by hydrolysis of the corresponding nitrile [—CN], using for example a base such as sodium hydroxide in a refluxing alcoholic solvent, such as ethanol.

In a further example, —OR⁹ or —OR²⁰ groups [where R⁹ or R²⁰ each represents an alkyl group such as methyl group] in compounds of formula (1) may be cleaved to the corresponding alcohol —OH by reaction with boron tribromide in a solvent such as a halogenated hydrocarbon, e.g. dichloromethane at a low temperature, e.g. around −78° C.

Alcohol [—OH] groups may also be obtained by hydrogenation of a corresponding —OCH₂R³⁰ group (where R³⁰ is an aryl group) using a metal catalyst, for example palladium on a support such as carbon in a solvent such as ethanol in the presence of ammonium formate, cyclohexadiene or hydrogen, from around ambient to the reflux temperature. In another example, —OH groups may be generated from the corresponding ester [e.g. CO₂Alk⁵ or CO₂R¹²] or aldehyde [—CHO] by reduction, using for example a complex metal hydride such as lithium aluminium hydride or sodium borohydride in a solvent such as methanol. Alternatively an alcohol may be prepared by reduction of the corresponding acid [—CO₂H], using for example lithium aluminium hydride in a solvent such as tetrahydrofuran.

In another example, alcohol —OH groups in the compounds may be converted to a corresponding —OR⁹ or —OR²⁰ group by coupling with a reagent R⁹OH or R²⁰OH in a solvent such as tetrahydrofuran in the presence of a phosphine, e.g. triphenylphosphine and an activator such as diethyl-, diisopropyl-, or dimethylazodicarboxylate.

Aldehyde [—CHO] groups may be obtained by oxidation of a correspondiing alcohol using well known conditions. For example using an oxidising agent such as a periodinane e.g Dess Martin, in a solvent such as a halogenated hydrocarbon, e.g. dichloromethane. An alternative oxidation may be suitably activating dimethyl sulfoxide using for example, oxalyl chloride, followed by—addition of an alcohol, and subsequent quenching of the reaction by the addition of an amine base, such as triethylamine. Suitable conditions for this reaction may be using an appropriate solvent, for example, a halogenated hydrocarbon, e.g. dichloromethane at −78° C. followed by subsequent warming to room temperature.

α,β-Unsaturated aldehydes, for example, of formula OHCD, where D is alkenyl or cycloalkenyl, may be prepared by hydrolysis of a corresponding allylic nitro compound. This may be achieved, for example, by treatment of the allylic nitro compound with a base, such as sodium methoxide or potassium tert-butoxide, followed by addition of a buffered aqueous titanium trichloride solution. The allylic nitro compound may be prepared by nucleophilic addition of nitromethane to the corresponding ketone, followed by elimination of water.

Suitable conditions for this reaction may be refluxing in toluene under Dean Stark conditions, in the presence of an amine base, such as N,N-dimethylethylene diamine. It will be appreciated that these aldehydes may be used in reductive alkylations to give compounds of formula (1), where Alk² is —CH₂— and L² is a covalent bond, using the conditions described herein.

Aminosulphonylamino [—NHSO₂NHR²⁰] groups in the compounds may be obtained, in another example, by reaction of a corresponding amine [—NH₂] with a sulphamide R²⁰NHSO₂NH₂ in the presence of an organic base such as pyridine at an elevated temperature, e.g. the reflux temperature.

In another example compounds containing a —NHCSR²⁰ or —CSNHR¹⁰, may be prepared by treating a corresponding compound containing a —NHCOR²⁰ or —CONHR¹⁰ group with a thiation reagent, such as Lawesson's Reagent or P₂S₅, in an anhydrous solvent, for example a cyclic ether such as tetrahydrofuran, at an elevated temperature such as the reflux temperature.

In a further example amine (—NH₂) groups may be alkylated using a reductive alkylation process employing an aldehyde and a borohydride, for example sodium triacetoxyborohyride or sodium cyanoborohydride, in a solvent such as a halogenated hydrocarbon, e.g. dichloromethane, a ketone such as acetone, or an alcohol, e.g. ethanol, where necessary in the presence of an acid such as acetic acid at around ambient temperature. Amines of formula —NH(CH₃) may be prepared by reacting the corresponding amine [—NH₂] with aqueous formaldehyde and cyclopentadiene in a suitable solvent such as water followed by reaction with trifluoroacetic acid and triethylsilane in a suitable halogenated hydrocarbon, e.g. dichloromethane to give the desired amine.

In a further example, amine [—NH₂] groups in compounds of formula (1) may be obtained by hydrolysis from a corresponding imide by reaction with hydrazine in a solvent such as an alcohol, e.g. ethanol at ambient temperature.

In another example, a nitro [—NO₂] group may be reduced to an amine [—NH₂], for example by catalytic hydrogenation using for example hydrogen in the presence of a metal catalyst, for example palladium on a support such as carbon in a solvent such as an ether, e.g. tetrahydrofuran or an alcohol e.g. methanol, or by chemical reduction using for example a metal, e.g. tin or iron, in the presence of an acid such as hydrochloric acid.

In a further example amine (—CH₂NH₂) groups in compounds of formula (1) and intermediates thereto may be obtained by reduction of nitrites (—CN), for example by catalytic hydrogenation using for example hydrogen in the presence of a metal catalyst, for example palladium on a support such as carbon, or Raney® nickel, in a solvent such as an ether e.g. a cyclic ether such as tetrahydrofuran or an alcohol, e.g. methanol or ethanol, optionally in the presence of ammonia solution at a temperature from ambient to the reflux temperature, or by chemical reduction using for example a metal hydride, e.g. lithium aluminium hydride, in a solvent such as an ether, e.g. a cyclic ether such as tetrahydrofuran, at a temperature from 0° C. to the reflux temperature.

Aromatic halogen substituents in the compounds may be subjected to halogen-metal exchange with a base, for example a lithium base such as n-butyl or t-butyl lithium, optionally at a low temperature, e.g. around −78° C., in a solvent such as tetrahydrofuran and then quenched with an electrophile to introduce a desired substituent. Thus, for example, a formyl group may be introduced by using dimethylformamide as the electrophile; a thiomethyl group may be introduced by using dimethyldisulphide as the electrophile.

In another example, sulphur atoms in the compounds, for example when present in a linker group L² or L³ may be oxidised to the corresponding sulphoxide or sulphone using an oxidising agent such as a peroxy acid, e.g. 3-chloroperoxybenzoic acid, in an inert solvent such as a halogenated hydrocarbon, e.g. dichloromethane, at around ambient temperature.

N-oxides of compounds of formula (1) may be prepared for example by oxidation of the corresponding nitrogen base using an oxidising agent such as hydrogen peroxide in the presence of an acid such as acetic acid, at an elevated temperature, for example around 70° C. to 80° C., or alternatively by reaction with a peracid such as peracetic acid in a solvent, e.g. dichloromethane, at ambient temperature.

Nitrogen quaternised derivatives of compounds of formula (1) may be formed by reaction of a compound of formula (1) with an alkylating agent such as an alkyl halide, e.g. methyl or ethyl iodide or a benzyl halide such as benzyl bromide in a solvent such as a halogenated hydrocarbon, e.g. dichloromethane or an alcohol, e.g. methanol or ethanol or a mixture of such solvents at for example ambient temperature.

Salts of compounds of formula (1) may be prepared by reaction of a compound of formula (1) with an appropriate base or acid in a suitable solvent or mixture of solvents e.g. an organic solvent such as an ether e.g. diethylether, or an alcohol, e.g. ethanol or an aqueous solvent using conventional procedures. Salts of compounds of formula (1) may be exchanged for other salts by use of conventional ion-exchange chromatography procedures.

Where it is desired to obtain a particular enantiomer of a compound of formula (1) this may be produced from a corresponding mixture of enantiomers using any suitable conventional procedure for resolving enantiomers.

Thus for example diastereomeric derivatives, e.g. salts, may be produced by reaction of a mixture of enantiomers of formula (1) e.g. a racemate, and an appropriate chiral compound, e.g. a chiral base. The diastereomers may then be separated by any convenient means, for example by crystallisation and the desired enantiomer recovered, e.g. by treatment with an acid in the instance where the diastereomer is a salt.

In another resolution process a racemate of formula (1) may be separated using chiral High Performance Liquid Chromatography. Alternatively, if desired a particular enantiomer may be obtained by using an appropriate chiral intermediate in one of the processes described above.

Chromatography, recrystallisation and other conventional separation procedures may also be used with intermediates or final products where it is desired to obtain a particular geometric isomer of the invention.

The following Examples illustrate the invention. All temperatures are in ° C. Where experimental detail is not given for the preparation of a reagent it is either commercially available, or it is known in the literature, for which the CAS number is quoted. The following abbreviations are used:

| | |
|---|---|
| NMM—N-methylmorpholine; | EtOAc—ethyl acetate; |
| MeOH—methanol; | BOC—butoxycarbonyl; |
| DCM—dichloromethane; | AcOH—acetic acid; |
| DIPEA—diisopropylethylamine; | EtOH—ethanol; |
| Pyr—pyridine; | DMF—N,N-dimethyl- |
| DMSO—dimethylsulphoxide; | formamide; |
| Et₂O—diethylether; | iPr—isopropyl; |
| THF—tetrahydrofuran; | Me—methyl; |
| Et₃N—triethylamine; | RT—room temperature; |
| DMF—N,N-dimethylformamide | LiAlH₄—lithium aluminium |
| EDC.HCl—1-(3-Dimethylaminopropyl)- | hydride; |
| 3-ethylcarbodimide hydrochloride; | |
| DMAP—4-dimethylaminopyridine | |
| NMRs were obtained at 400 MHz unless | |
| otherwise indicated. | |

Intermediate 1

1-Cyclooct-1-enylmethylpiperidin-4-ylamine dihydrochloride

(Boc-4-amino)piperidine.HCl (4.56 g), triethylamine (2.6 ml), molecular sieves (1 g) and THF (200 ml) were combined under a nitrogen atmosphere at RT. Cyclooct-1-enecarbaldehyde (CAS No. 6038-12-6) (4 g) was added and the reaction mixture stirred for 15 mins. NaBH(OAc)₃ (8 g) was added and the reaction was stirred at RT for 16 h. The reaction mixture was then evaporated in vacuo and the resulting residue was taken up in DCM (200 ml), washed with H₂O (2×100 ml), saturated aqueous NaHCO₃ (100 ml), dried (MgSO₄) and evaporated in vacuo to give a yellow oil (6.94 g). The yellow oil was dissolved in MeOH (50 ml) and 1N HCl in Et₂O (110 ml) was added. The reaction mixture was stirred at RT for 2 h. The resulting precipitate was filtered and washed with Et₂O to give the title compound as a white solid. Evaporation of the filtrate in vacuo and trituration with Et₂O gave a second crop of the title compound as a beige solid. MS 223 (M+H).

Intermediate 2

(2-Methylsulfanylbenzoxazol-5-yl)acetic acid

(3-Amino-4-hydroxyphenyl)acetic acid (4 g), EtOH (50 ml) and potassium ethyl xanthate (4.6 g) were combined and heated to reflux for 5 h. The reaction mixture was allowed to cool to RT, iodomethane (3.3 ml) was added and the whole stirred for 16 h. The EtOH was removed by evaporation in vacuo and the residue was partitioned between EtOAc (250 ml) and H₂O (250 ml). The organic layer was washed with H₂O (250 ml), brine (250 ml), dried (MgSO₄) and evaporated in vacuo to give the title compound as a beige solid. MS 224 (M+H).

Intermediate 3

Benzofuran-3-yl-acetic acid ethyl ester

3-Coumaranone (1.50 g) and (carboethoxyrnethylene)triphenylphosphorane (5.84 g) were refluxed in toluene (50 ml) for 48 hours. The toluene was removed in vacuo and the residue was purified by Flash chromatography to give the title compound as a near colourless oil (1.75 g) R_{f}=0.18 (10:1 hexane/EtOAc); MS 205 (M+H).

Intermediate 4

Benzofuran-3-yl-acetic acid

To a stirring solution of Intermediate 3 (1.00 g) in methanol (20 ml) was added a solution of lithium hydroxide (2.05 g) in water. The mixture was stirred for 2 hours before diluting with water (50 ml) and washing with hexane (20 ml). The aqueous layer was acidified (HCl, pH1) and extracted with EtOAc (4×50 ml). The combined organic layers were washed with water (2×25 ml), brine (25 ml), dried (MgSO₄) and evaporated in vacuo to give the title compound as an off-white solid. MS 177 (M+H).

Intermediate 5

3-Benzofuran-4-yl-propionic acid

To a stirred solution of 3-(1-benzofuran-4-yl)prop-2-enoic acid (CAS 209256-70-2, 382 mg) in ethanol (30 ml) was added palladium on charcoal (10%, 10 mg). The mixture was hydrogenated for 4 hrs before the catalyst was filtered and washed with ethanol. The filtrate was evaporated in vacuo_and the residue was purified by flash chromatography to give the title compound as a near colourless oil (113 mg) R_{f}=0.28 (1:1 hexane/EtOAc); MS 191 (M+H).

Intermediate 6

Benzo[b]thiophen-3-yl-acetic acid

3-Benzothiopheneacetonitrile (CAS No. 3216-48-6) (5.0 g) and sodium hydroxide (8 g) was heated to reflux in ethanol/water (3:1, 80 ml) for 3 hrs. The mixture was cooled evaporated in vacuo and acidified. The solid precipitate was collected, washed with water and dried to give the title compound as a beige solid R_{f} 0.40 (Ether).

Intermediate 7

4-(2-Benzofuran-4-yl-acetylamino)piperidine-1-carboxylic acid tert-butyl ester

2-(Benzofuran-4-yl)acetic acid (2.22 g), Boc-(4-amino)piperidine.HCl (2.99 g) and DCM (50 ml) were combined under a nitrogen atmosphere at RT. Triethylamine (3.52 ml) was added, followed by DMAP (10 mg) and EDC.HCl 3.62 g) and the reaction mixture stirred for 16 h at RT. 1M NaOH (50 ml) was added, organic layer was separated and washed with 1M NaOH (50 ml) and brine (50 ml), dried (MgSO₄) and evaporated in vacuo to give a yellow solid (5.85 g). The crude residue was dissolved in EtOAc and washed through a pad of silica with 15% DCM, EtOAc. The filtrate was collected and evaporated to dryness in vacuo to give the title compound as a cream solid (4.11 g). R_{f}=0.27 (50% EtOAc/hexane); δH (CDCl₃) 7.20 (1H, d), 7.50 (1H, d), 7.30 (1H, t), 7.10 (1H, d), 6.80 (1H, d), 5.25 (1H, d), 4.10−3.90 (3H, m), 3.85 (2H, s), 2.80 (2H, t), 1.80 (2H, dd), 1.45 (9 h, s), 1.20−1.00 (2H, m); MS 359 (M+H).

Intermediate 8

2-Benzofuran-4-yl -N-piperidin-4-yl-acetamide hydrochloride

Intermediate 7 (3.39 g) was dissolved in MeOH (50 ml) under a nitrogen atmosphere, 1M HCl (in Et₂O) (95 ml) was added and reaction stirred at RT for 3 h. Reaction mixture was evaporated to dryness in vacuo to yield the title compound as a cream foam (2.3 g). MS 259 ((M−HCl)+H).

Intermediate 9

2-Cyclohexyl-propionaidehyde

Oxalyl chloride (0.55 ml) and DMSO (0.90 ml) were added to cooled DCM (15 ml) at −78° C. and the reaction stirred for 5 min. 2-Cyclohexyl-1-propanol (0.50 ml) was then added dropwise and the reaction stirred at −78° C. for 1.5 h. Triethylamine (3.1 ml) was then added and the reaction allowed to reach RT and stirred for a further 1.5 h. The reaction mixture was partitioned between DCM (40 ml) and water (40 ml). The aqueous layer was back-extracted with DCM (40 ml) and the combined organics washed with water (2×40 ml), dried (MgSO₄) and evaporated to dryness in vacuo to yield a yellow oil (721 mg). The product was purified by flash chromatography (5% EtOAc/hexane) to yield the title compound as a yellow oil (272 mg). R_{f}=0.39 (5% EtOAc/hexane); δH (CDCl₃) 9.65 (1H, d), 2.35−2.15 (1H, m), 1.90−1.60 (7H, m), 1.45−1.10 (4H, m), 1.05 (3H, d).

Intermediate 10

3-(2-Benzofuran-4-yl-acetylamino)piperidine-1-carboxylic acid tert-butyl ester

2-(Benzofuran-4-yl)acetic acid (0.77 g) and (±)-(3-amino-1-Boc)piperidine. (0.88 g) were dissolved in DCM (15 ml) under a nitrogen atmosphere. Triethylamine (1.23 ml) was added, followed by DMAP (10 mg) and EDC.HCl (1.26 g) and the reaction mixture stirred for 16 h at RT. DCM (25 ml) was added, the reaction mixture washed with 1M NaOH (2×30 ml) and brine (30 ml), dried (MgSO₄) and evaporated in vacuo to give a cream solid. The product was purified by flash chromatography (5% MeOH, DCM) to yield the title compound as a cream solid (0.94 g). R_{f}=0.74 (10% MeOH, DCM); δH (CDCl₃) 7.65 (1H, d), 7.45 (1H, d), 7.30−7.25 (1H, m), 7.10 (1H, d), 6.80 (1H, d), 5.45 (1H, br s), 4.00−3.85 (1H, m), 3.80 (2H, s), 3.50 (1H, d), 3.35−3.00 (3H, m), 1.75−1.60 (2H, m), 1.40 (9H, s); MS 359.0 (MH+), 381.2 (M+Na).

Intermediate 11

2-Benzofuran-4-yl-N-piperidin-3-yl-acetamide hydrochloride

Intermediate 10 (0.94 g) was dissolved in MeOH (50 ml) under a nitrogen atmosphere, 1M HCl (in Et₂O) (95 ml) was added and reaction stirred at RT for 16 h. Reaction mixture was evaporated to dryness in vacuo to yield the title compound as a pale yellow solid (0.93 g) MS 259.3 ((M−HCl)+H).

Intermediate 12

3-(3,4-Dichlorophenyl)propan-1-ol

3-(3,4-Dichlorophenyl)propionic acid (1.38 g) was dissolved in THF (40 ml) under a nitrogen atmosphere and cooled to 0° C. LiAlH₄ (0.24 g) was added and the reaction stirred for 22 h at RT. The reaction was quenched by addition of EtOAc (2 ml), H₂O (1.5 ml) and 1M NaOH (0.5 ml) and the mixture was stirred for a further 30 min. The reaction mixture was then filtered through Celite® and concentrated in vacuo. H₂O (10 ml) was added and then extracted with DCM (2×10 ml). Combined organics were dried (MgSO₄) and evaporated to dryness in vacuo. The product was purified by flash chromatography (5% MeOH, DCM) to yield the title compound as a colourless oil (0.63 g). R_{f}=0.50 (10% MeOH, DCM); δH (CDCl₃) 7.40−7.25 (2H, m), 7.00 (1H, dd), 3.65 (2 h, t), 2.70 (2H, t), 1.90−1.80 (2H, m).

Intermediate 13

2-Methyl-3-phenylpropan-1-ol

α-Methylhydrocinnamic acid (1.25 g) was dissolved in THF (20 ml) under a nitrogen atmosphere and LiAlH₄ (1M solution in THF) (8.4 ml) was added and reaction stirred for 16 h at RT. The reaction was quenched and worked up as described for intermediate 12 to yield the title compound as a colourless oil (1.09 g ). The product was purified by flash chromatography (50% EtOAc, hexane) to yield the title compound as a colourless oil (0.55 g). R_{f}=0.64 (10% MeOH, DCM); δH (CDCl₃) 7.30−7.10 (5H, m), 3.60−3.40 (2 h, m), 2.80−2.70 (1H, m), 2.45−2.35 (1H, m), 2.00−1.85 (1H, m), 0.95 (3H, d).

Intermediate 14

3-(3,4-Dichlorophenyl)propanal

Intermediate 12 (0.51 g) was dissolved in DCM (15 ml) under an atmosphere of nitrogen. Dess-Martin periodinane (1.2 g) was added and reaction stirred at RT for 6 h. 1M NaOH (15 ml) added and stirred for a further 30 min. The reaction mixture was separated, and the aqueous back-extracted with DCM (2×20 ml). The combined organics were washed with brine (20 ml) and dried (MgSO₄) and evaporated to dryness in vacuo to yield the title compound as a yellow oil (0.38 g). R_{f}=0.87 (10% MeOH, DCM); δH (CDCl₃) 9.80 (1H, s), 7.40−7.25 (2H, m), 7.00 (1H, dd), 2.95−2.85 (2H, m), 2.80−2.75 (2H, m).

Similarly prepared was:

Intermediate 15

2Methyl-3-phenylpropanal

From Intermediate 13 (0.31 g) as a colourless oil. R_{f}=0.89 (50% EtOAc/hexane).

### EXAMPLE 1

2-Benzofuran-4-yl-N-(1-cyclooct-1-enyimethylpiperidin-4-yl)acetamide

2-(1-Benzofuran-4-yl)acetic acid (200 mg), EDC.HCl (326 mg), Intermediate 1 (335 mg), DMAP (10 mg), Et₃N (0.63 ml) and DCM (20 ml) were combined under a nitrogen atmosphere and stirred at RT for 16 h. The reaction mixture was then diluted with DCM (20 ml), washed with 1N aqueous NaOH solution (20 ml) and evaporated in vacuo onto silica. Purification by flash chromatography gave the title compound as an off white solid. R_{f}=0.4 (10% MeOH, CH₂Cl₂); δH (d₆-DMSO) 8.1(1H, d), 7.95 (1H, s), 7.45 (1H, m), 7.25 (1H, m), 7.10 (1H, m), 7.05 (1H, s), 5.45 (1H, t), 3.65 (2H, s), 3.50 (1H, m), 3.20 (2H, s), 2.80−2.65 (4H, m), 2.20−1.20 (16H, m).

Similarly prepared were:

### EXAMPLE2

2-(2-Chloro-benzothiazol-6-yl)-N-(1-cyclooct-1-enylmethyl-piperidin n-4-yl)-acetamide

From (2-chloro-benzothiazol-6-yl)acetic acid (77 mg) and Intermediate 1 as an off white solid. R_{f}=0.375 (10% MeOH, DCM); MS 432, 434 (M+H).

### EXAMPLE3

2-(5-Chloro-benzo[b]thiophen-3-yl)-N-(1-cyclooct-1-enylmethylpiperidin-4-yl)acetamide

From 5-chlorobenzo[b]thiophene-3-acetic acid (78 mg) and Intermediate 1 as an off white solid. R_{f}=0.44 (10% MeOH, DCM); MS 431, 433 (M+H).

### EXAMPLE4

N-(1-cyclooct-1-enylmethylpiperidin-4-yl)-2-(2-methylsulfanylbenzoxazol-5-yl)-acetamide

From Intermediate 2 (75 mg) and Intermediate 1 as a pale yellow solid. R_{f}=0.375 (10% MeOH, DCM); MS 428 (M+H).

### EXAMPLE5

2-Benzofuran-3-yl-N-(1-cyclooct-1-enyimethylpiperidin-4-yl)acetamide

From Intermediate 4 (66 mg) and Intermediate 1 as an off-white solid R_{f}=0.11 (5% MeOH/DCM); MS 381 (M+H).

### EXAMPLE 6

3-Benzofuran-4-yl-N-(1-cyclooct-1-enyimethylpiperidin-4-yl)propionamide

From intermediate 5 (110 mg) and Intermediate 1 as a glassy solid R_{f}=0.28 (7% MeOH/DCM); MS 395 (M+1).

### EXAMPLE 7

N-(1-Cyclooct-1-enylmethylpiperidine-4-yl)-2-(1-methyl-1 H-indol-3-yl)acetimidate

From 3-(N-methylindole)-acetic acid (32 mg) and Intermediate 1 as a off-white solid R_{f}=0.40 (10% MeOH/DCM); MS 394 (M+H).

### EXAMPLE 8

2-Benzothiophen-3-yl-N-(1-cyclooct-1-enylmethylpiperidin-4-yl)acetamide

From intermediate 6 (65 mg) and Intermediate 1 as a tan solid R_{f}=0.45 (10% MeOH/DCM); MS 397 (M+H); δH (CDCl₃) 7.87-7.93 (1H, m), 7.70−7.77 (1H, m), 7.36-7.43 (2H, m), 7.34 (1H, s), 5.44 (1H, t), 5.33 (1H, bd), 3.80 (2H, s), 3.73-3.87 (1H, m), 2.75 (2H, s), 2.55-2.70 (2H, m), 2.04-2.19 (4H, m), 1.90-2.04 (2H, m), 1.72-1.83 (2H, m), 1.37-1.70 (8H, m), 1.20-1.35 (2H, m).

### EXAMPLE 9

3-Benzo[b]thiazol-2-yl-N-(cyclooct-1-enylmethylpiperidin-4-yl)propionamide

From 3-(benzo[b]thiazol-2-yl)propionic acid (35 mg) and Intermediate 1 as a colourless oil. R_{f}=0.37 (10% MeOH/DCM); MS 412 (M+H).

### EXAMPLE 10

2-Benzofuran-2-yl-N-(1-cyclooct-1-enylmethyl-piperidin-4-yl)acetamide

From 2-(benzofuran-2-yl)acetic acid (CAS No 62119-70-4, 100 mg) and Intermediate 1 as a colourless oil R_{f}=0.53 (15% MeOH/DCM);MS 381 (M+H).

### EXAMPLE 11

4-(2-Benzofuran-4-yl-ethanoylamino)-1-cyclooct-1-enylmethyl-1-ethyl-piperidinium iodide

The compound of Example 1 (71 mg) and iodoethane (5 ml) were combined and heated to 75° for 70 h. The reaction mixture was evaporated in vacuo to give the title compound as a brown solid (mixture of trans/cis isomers; ratio 5:1). MS 409 (M+ for salt); δH (CDCl₃) major isomer: 8.00−7.90 (1H, br s), 7.60−7.00 (5H, m), 6.00 (1H, t), 4.30−3.10(11H, m), 2.50−1.20 (19H, m).

### EXAMPLE 12

2-Benzofuran-4-yl-N-[1-(6,6-Dimethyl-bicyclo[3.1.1]hept-2-en-2-ylmethyl)piperidin-4-yl]acetamide

Intermediate 8 (100 mg), (1R)-(−)myrtenal (0.052 ml), triethylamine (0.05 ml) and powdered molecular sieves (4 Å) were combined in tetrahydrofuran (10 ml) under nitrogen The reaction mixture was stirred for 30 min before addition of sodium triacetoxyborohydride (144 mg). After stirring for 20 h the reaction was quenched by addition of sat. NaHCO₃ and extracted with DCM (3×20 ml). The combined organic extracts were washed with brine (20 ml), dried (MgSO₄) and evaporated in vacuo. Purification by flash chromatography gave the title compound as a white solid (68 mg). R_{f}=0.22 (5% MeOH/DCM); δH (CDCl₃) 7.70 (1H, d), 7.52 (1H, d), 7.40 (1H; d), 7.12 (1H, m), 6.85 (1H, m), 5.36 (1H, m), 5.28 (1H, m), 3.83 (2H, s), 3.80−3.68 (1H, m), 2.89−2.62 (4H, m), 2.40−2.24 (4H, m), 2.15−1.95 (4H, m), 1.89−1.80 (3H, m), 1.38−1.19 (2H, m), 1.30 (3H, s), 1.08 (1H, d), 0.80 (3H, s); MS 393 (M+H).

Similarly prepared were:

### EXAMPLE 13

2-Benzofuran-4-yl-N-[1-(2-ethyl-hex-2-enyl)piperidin-4-yl]acetamide

From 2-ethyl-2-hexenal (43 mg) and Intermediate 8 as a colourless oil. R_{f}=0.16 (5% MeOH/DCM); MS 369 (M+H).

### EXAMPLE 14

2-Benzofuran-4-yl-N-[1-(3,5,5-trimethylhexyl)piperidin-4-yl]acetamide

From 3,5,5-trimethylhexanal (0.088 ml) and Intermediate 8 as a white solid R_{f}=0.37 (10% MeOH/DCM); MS 385 (M+H).

### EXAMPLE 15

2-Benzofuran-4-yl-N-[1-((S)-6,6-Dimethyl-bicyclo[3.1.1 ]hept-2-ylmethyl)piperidin-4-yl]acetamide

From (1S, 2S, 5S)-(−)-myrtanal (CAS No 128301-02-01, 77 mg) and Intermediate 8 as a white solid R_{f} 0.48 (10% MeOH/DCM); MS 395 (M+H).

### EXAMPLE 16

2-Benzofuran-4-yl-N-[1-(2-cyclohexylpropyl)piperidin-4-yl]acetamide

From Intermediate 9 (73 mg) and Intermediate 8 as a white solid (107 mg).

R_{f}=0.50 (10% MeOH/DCM); δH (CDCl₃) 7.65 (1H, d), 7.50 (1H, d), 7.25 (1H, d), 7.05 (1H, d), 6.80 (1H, d), 5.35 (1H, br d), 3.85−3.70 (3H, m), 3.50 (2H, s), 2.85−2.60 (2H, m), 2.30−0.90 (18H, m), 0.80 (3H, d); MS 383.4 (M+H).

### EXAMPLE 17

2-Benzofuran-4-yl-N-[1-(5-fluoro-2,3-dihydrobenzofuran-2-ylmethyl)piperidin-4-yl]acetamide

Intermediate 8 (0.14 g) was partitioned between DCM (3×10 ml) and IM sodium hydroxide (20 ml). The organic phase was washed with brine (20 ml), dried (MgSO₄) and cocncentrated in vacuo to give the amine free base for use in the following reaction:

2-Benzofuran-4-yl-N-piperidin-4-ylacetamide (122 mg), 2-bromomethyl-5-fluorocoumaran (100 mg), triethylamine (0.10 ml) and tetrabutylammonium iodide (10 mg) were combined in DMF (5 ml) under nitrogen. The reaction mixture was stirred for 60 h, diluted with DCM (25 ml) and washed with H₂O (4×10 ml) and brine (10 ml), dried (MgSO₄) and evaporated in vacuo. Purification by flash chromatography gave the title compound as a yellow solid (60 mg). R_{f}=0.16 (5% MeOH, CH₂Cl₂); δH (CDCl₃) 7.68 (1H, m), 7.48 (1H, m), 7.30 (1H, m), 7.10 (1H, m), 6.88−6.72 (3H, m), 6.65 (1H, m), 5.22 (1H, m), 4.90−4.80 (1H, m), 3.80 (2H, m), 3.25−3.15 (1H, m), 2.92−2.65 (3H, m), 2.50−2.42 (1H, m), 2.20−2.10 (2H, m), 1.85−1.78 (4H, m), 1.30−1.22 (2H, m), MS 409 (M+H).

Similarly prepared was:

### EXAMPLE 18

2-Benzofuran-4-yl-N-[1-(2-phenoxyethyl)piperidin-4-yl]acetamide

From β-bromophenetole (75mg) and 2-benzofuran-4-yl-N-piperidin-4-ylacetamide as a white solid R_{f} 0.2 (15% MeOH/DCM) MS 379 (M+H).

### EXAMPLE 19

2-Benzofuran-4-yl-N-[1-(3-phenylpropyl)piperidin-3-yl]acetamide

Intermediate 11 (132 mg), 3-phenylpropionaldehyde (0.1 ml) and triethylamine (0.07 ml) were combined in tetrahydrofuran (10 ml) under nitrogen. The reaction mixture was stirred for 10 min before addition of sodium triacetoxyborohydride (293 mg). After stirring for 20 h the reaction was quenched by addition of 1M NaOH (30 ml) and extracted with DCM (3×20 ml). The combined organic extracts were washed with brine (20 ml), dried (MgSO₄) and evaporated in vacuo. Purification by flash chromatography gave the title compound as a white solid (90 mg).

R_{f}=0.48 (10% MeOH, DCM); δH (CDCl₃) 7.55 (1H, d), 7.40−7.00 (7H, m), 6.80 (1H, s), 6.15 (1H, br s), 4.05 (1H, br s), 3.80 (2H, s), 2.50−1.90 (6H, m), 1.80−1.20 (9H, m); MS 377.3 (MH+).

### EXAMPLE 20

2-Benzofuran-4-yl-N-(1-benzylpiperidin-4-yl)acetamide

2-(1-Benzofuran-4-yl)acetic acid (500 mg), EDC.HCl (814 mg), 4-amino-N-benzylpiperidine (0.58 ml), DMAP (10 mg), and DCM (50 ml) were combined under a nitrogen atmosphere and stirred at RT for 16 h. The reaction mixture was then diluted with DCM (50 ml), washed with 1N aqueous NaOH solution (40 ml), brine (40 ml) and evaporated in vacuo. Purification by flash chromatography gave the title compound as a white solid (950 mg). R_{f}=0.31 (10% MeOH, DCM); δH (CDCl₃) 7.65 (1H, d), 7.48 (1H, d), 7.38−7.25 (6H, m), 7.19 (1H, d), 6.80 (1H, d), 5.30−5.25 (1H, m), 3.85−3.78 (3H, m), 3.48 (2H, s), 2.82−2.70 (2H, m), 2.15−2.10 (2H, m), 1.96−1.80 (2H, m), 1.40−1.30 (2H, m); MS 349 (M+H).

### EXAMPLE 21

3-Benzofuran-2-yl-N-[1-(3,4-dichlorobenzyl)piperidin-4-yl]propionamide

3-(Benzofuran-2-yl)propionic acid (CAS No 21683-86-3, 612 mg) in DCM (10 ml) was treated with oxalyl chloride (0.56 ml) and DMF (1 drop). The mixture was stirred for 18 hrs and the solvents were removed in vacuo. The residue was dissolved in DCM (10 ml) and 1-(3,4-dichlorobenzyl)piperidin-4-ylamine dihydrochloride (CAS No 57645-61-1, 834mg) and triethylamine (1.4 ml) was added. The mixture was stirred for 1.5 hr before diluting with DCM and washed with 1N aqueous NaOH solution (20 ml) and evaporated in vacuo onto silica. Purification by flash chromatography gave the title compound as an off white solid. R_{f}=0.45 (10% MeOH/DCM); ); δH (CDCl₃) 7.50−7.10 (7H, m), 6.40 (1H, s), 5.35 (1H, d), 3.90−3.70 (1H, m), 3.35 (2H, s), 3.10 (2H, t), 2.70−2.55 (4H, m), 2.10 (2H, t), 1.85 (2H, d), 1.45−1.20 (2H, m); MS 432 (M+H).

### EXAMPLE 22

2-Benzofuran-4-yl-N-[1-(3-phenylpropylpiperidin-4-yl]acetamide

Intermediate 8 (100 mg), 3-phenylpropionaldehyde (0.05 ml), triethylamine (0.05 ml) and powdered molecular sieves (4 Å) were combined in tetrahydrofuran (10 ml) under nitrogen. The reaction mixture was stirred for 30 min before addition of sodium triacetoxyborohydride (144 mg). After stirring for 20 h the reaction was quenched by addition of sat. NaHCO₃ and extracted with DCM (3×20 ml). The combined organic extracts were washed with brine (20 ml), dried (MgSO₄) and evaporated in vacuo. Purification by flash chromatography gave the title compound as a white solid (97 mg). R_{f}=0.29 (10% MeOH, CH₂Cl₂); δH (CDCl₃) 7.67 (1H, m), 7.48 (1H, d), 7.28 (3H, m), 7.10 (4H, m), 6.80 (1H, m), 5.26 (1H, d), 3.78 (3H, m), 2.75 (2H, m), 2.60 (2H, m), 2.30 (2H, m), 2.10−2.00 (2H, m), 1.83−1.72 (4H, m), 1.35−1.22 (2H, m). MS 377 (M+H).

Similarly prepared were:

### EXAMPLE 23

2-Benzofuran-4-yl-N-[3,4-dichlorobenzyl)piperidin-4-yl]acetamide, formate salt

From 3,4-dichlorobenzaldehyde (89 mg) and Intermediate 8 with purification by reverse phase preparative HPLC using a 25 cm×21.2 mm Phenomenex Luna C18 (2) (5 u) column and a mobile phase of aqueous formic acid (0.1% v/v) and acetonitrile under gradient conditions form 5% to 75% acetonitrile. The title compound was obtained as a white solid. R_{f}=0.47 (10% MeOH/DCM); MS 418 [(M+H)-formate].

### EXAMPLE 24

2-Benzofuran-4-yl-N-{1-[3-(3,4-dichloro-phenyl)-propyl]-piperidin-4-yl}-acetamide

From Intermediate 14 (142 mg) and Intermediate 8 as a white solid (130 mg).

R_{f}=0.13 (10% MeOH, 89% DCM, 1% NH_{3(aq.))}, δH (CDCl₃) 7.65 (1H, d), 7.45 (1H, d), 7.2.35−7.15 (3H, m), 7.10 (1H, d), 6.95 (1H, d), 6.80 (1H, d), 5.25 (1H, br d), 3.85−3.70 (3H, m), 2.65 (2H, br d), 2.55 (2H, t), 2.25 (2H, t), 2.00 (2H, t), 1.85−1.55 (4H, m,), 1.35−1.15 (2H, m), MS 445.3 (M+H).

### EXAMPLE 25

2-Benzofuran-4-yl-N-[1-(2-methyl-3-phenyl-propyl)-piperidin-4-yl]acetamide

From Intermediate 15 (40 mg) and Intermediate 8 as a white solid (40 mg). R_{f}=0.37 (10% MeOH, DCM); δH (CDCl₃) 7.65 (1H, d), 7.40 (1H, d), 7.20−7.15 (5H, m), 7.10 (2H, d), 6.80 (1H, d), 5.25−5.15 (1H, m), 3.80 (2H, s), 2.80−2.50 (3H, m), 2.30−2.20 (1H, m), 2.15−1.70 (6H, m), 1.35−1.15 (3H, m), 0.80 (3H, d); MS 391.4 (M+H).

The following assays were used to demonstrate the potency and selectivity of the compounds according to the invention for inhibition of chemokine binding to CCR-3 receptors.

CCR-3 Calcium Assay

The following assay was performed using a FLIPR.

CHO cells stably transfected with CCR-3 were used in the assay. Thses were routinely passaged in RPMI 1640 with glutamine, non-essential amino-acids, 10% FCS and 0.4 mg/ml G-418 (the selection agent) at 37° with 5% CO₂, the cells were removed from the culture flask using non-enzymatic dissociation agent, washed, resuspended at 1.5×10⁵/ml in medium, dispensed into black-walled, clear-bottomed tissue culture plates at 200 μl/well and incubated overnight. The culture medium was replaced with 100 μl/well dye loading buffer (HBSS, 0.2% BSA, 1mM probenecid, 4 μM Fluo-4 and 0.08% pluronic acid). After 1-2 h incubation the loading buffer was removed and the plate washed leaving 100 μl/well of wash buffer (HBSS, 0.2% BSA, 1mM probenecid). Compounds were dissolved in DMSO the diluted 1:125 in wash buffer. The FLIPR was programmed to add diluted compound and after 2 mins diluted human recombinant eotaxin (final concentration of 10 nM). Inhibition was calculated as a function of maximum calcium response.

1. A compound of formula (1): 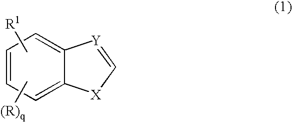
wherein:
q is zero or the integer 1, 2 or 3;
R which when present may be attached to any available carbon or nitrogen atom of the bicyclic heteroaromatic ring of formula (1) is an atom or group -L³(Alk³)_{w}L⁴(R⁸)ᵤ in which L³ and L⁴ which may be the same or different is each a covalent bond or a linker atom or group, w is zero or the integer 1, u is the integer 1, 2 or 3, Alk³ is an optionally substituted aliphatic or heteroaliphatic chain and R⁸ is a hydrogen or halogen atom (or a group selected from alkyl, —OR⁹ [where R⁹ is a hydrogen atom or an optionally substituted alkyl group], —SR⁹, —NR⁹R¹⁰, [where R¹⁰ is as just defined for R⁹ and may be the same or different]. —NO₂, —CN, —CO₂R⁹, —OCO₂R⁹, —CONR⁹R¹⁰, —OCONR⁹R¹⁰, —CSNR⁹R¹⁰, —COR⁹, —OCOR9, —N(R⁹)COR¹⁰, —N(R⁹)CSR¹⁰, —SO₂N(R⁹)(R¹⁰), —N(R⁹)SO₂R¹⁰, —N(R⁹)CON(R¹⁰)(R¹¹), [where R¹¹ is a hydrogen atom or an optionally substituted alkyl group], —N(R⁹)CSN(R¹⁰)(R¹¹), —N(R⁹)SO₂N(R¹⁰)(R¹¹) or an optionally substituted cycloaliphatic, heterocycloaliphatic, aromatic or heteroaromatic group provided that when w is zero and each of L³ and L⁴ is a covalent bond then u is the integer 1; or
when q is the integer 2 or 3 and two R groups are attached to adjacent carbon atoms of the heteroaromatic ring of formula (1) these may be joined together with the heteroaromatic ring carbon atoms to form an optionally substituted cycloaliphatic, heterocycloaliphatic, aromatic or heteroaromatic group;
X is an O atom or a S(O)ₘ atom or group in which m is zero or the integer 1 or 2 or an NR group;
Y is a N atom or a CR^{1a} group in which R^{1a} is a group R or a group R¹;
R¹ which may be on any available carbon atom of the bicyclic heteroaromatic ring of formula (1) is a hydrogen atom or a group -Alk¹L¹CyAlk²L²D in which Alk¹ is an optionally substituted C₁₋₃alkyl group, Alk² is an optionally substituted aliphatic or cycloaliphatic group, L¹ is a —CON(R⁵)—, —N(R⁵)CO—, —SO₂N(R⁵)— or —N(R⁵)SO₂— group in which R⁵ is a hydrogen atom or an optionally substituted aliphatic, heteroaliphatic, cycloaliphatic, polycycloaliphatic, heterocycloaliphatic, heteropolycycloaliphatic, aromatic, or heteroaromatic group, L² is a covalent bond or an —O— atom or —S(O)ₙ— atom or group in which n is zero or the integer 1 or 2 or —N(R⁷)— group in which R⁷ is a hydrogen atom or an optionally substituted aliphatic, heteroaliphatic, cycloaliphatic, polycycloaliphatic, heterocycloaliphatic, neteropolycycloaliphatic, aromatic, or heteroaromatic group, D is an optionally substituted aliphatic, heteroaliphatic, cycloaliphatic, polycycloaliphatic, heterocycloaliphatic, heteropolycycloaliphatic, aromatic, or heteroaromatic group and Cy is an optionally substituted heterocycloaliphatic ring of formula (A) or (B): 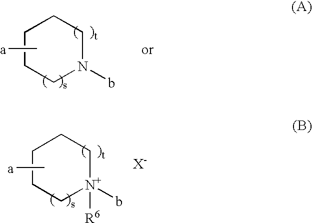
in which
a indicates the point of attachment of any available ring carbon in the ring Cy to the group L¹, b indicates the point of attachment to Alk², s and t which may be the same or different is each zero or the integer 1 or 2, provided that s+t is the integer 1, 2, 3 or 4, R⁶ is an optionally substituted alkyl group and X is a pharmaceutically acceptable counterion;
provided that at least one but not both of R¹ and R^{1a} is the group -Alk¹L¹CyAlk²L²D;
and the salts, solvates, hydrates, N-oxides thereof.

2. A compound of claim 1, wherein X is an O or S atom or a NR² group, where NR² is a NH, NCH₃ or NCH₂Ph group, where Ph is an optionally substituted phenyl ring.

3. A compound according to claim 2, wherein X is an oxygen atom.

4. A compound according to claims 1-3 of formula (2): 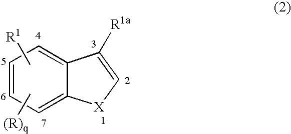
wherein the numbers 1 to 7 indicate the atom numbering of the heteroaromatic ring according to IUPAC nomenclature and R, q, R¹, R^{1a} and X are as generally and particularly defined herein for compounds of formula (1); and the salts, solvates, hydrates, N-oxides thereof.

5. A compound according to claims 1-4 wherein R^{1a} is a group Alk¹L¹CyAlk²L²D.

6. A compound according to claims 1-4 wherein R¹ is a group, -Alk¹L¹CyAlk²L²D and R^{1a} is an atom or group R.

7. A compound according to claim 6 wherein R¹ is attached to the carbon atom numbered 4.

8. A compound according to any preceding claim wherein q is zero or the integer 1 or 2, preferably zero.

9. A compound according to any preceding claim wherein Alk¹ in the substituent -Alk¹L¹CyAlk²L²D is an optionally substituted —CH₂— or —CH₂CH₂— group.

10. A compound according to any preceding claim wherein L¹ in the substituent -Alk¹L¹CyAlk²L²D is a —CON(R⁵)— group.

11. A compound according to any preceding claim wherein R¹ or the group R^{1a} is the group -Alk¹L¹CyAlk²L²D in which L¹ is the group —CON(R⁵)— in which R⁵ is a hydrogen atom or —CH₃ or —CH₂CH₃ group, L² is a covalent bond and Alk² is an optionally substituted —CH₂—, —CH(CH₃)— or —CH₂CH₂— group.

12. A compound according to any preceding claim wherein s and t in the heterocycloaliphatic ring Cy are each the integer 1.

13. A compound according to any preceding claim wherein D is an optionally substituted C₃₋₈cycloalkyl or C₃₋₈cycloalkenyl group.

14. A compound which is:
2-benzofuran-4-yl-N-(1-cyclooct-1-enylmethylpiperidin-4-yl)acetamide;
2-(2-chloro-benzothiazol-6-yl)-N-(1-cyclooct-1-enylmethyl-piperidin-4-yl)-acetamide;
2-(5-chloro-benzo[b]thiophen-3-yl)-N-(1-cyclooct-1-enylmethylpiperidin-4-yl)acetamide;
N-(1-cyclooct-1-enylmethylpiperidin-4-yl)-2-(2-methylsulfanylbenzoxazol-5-yl)-acetamide;
4-(2-benzofuran-4-yl-ethanoylamino)-1-cyclooct-1-enylmethyl-1-ethyl-piperidinium iodide;
and the salts, solvates, hydrates and N-oxides thereof.

15. A compound which is:
2-benzofuran-3-yl-N-(1-cyclooct-1-enylmethylpiperidin-4-yl)acetamide;
3-benzofuran-4-yl-N-(1-cyclooct-1-enylmethylpiperidin-4-yl)propionamide;
N-(1-cyclooct-1-enylmethylpiperidine-4-yl)-2-(1-methyl-1H-indol-3-yl)acetimidate;
2-benzothiophen-3-yl-N-(1-cyclooct-1-enylmethylpiperidin-4-yl)acetamide;
3-benzo[b]thiazol-2-yl-N-(cyclooct-1-enylmethylpiperidin-4-yl) propionamide;
2-benzofuran-2-yl-N-(1-cyclooct-1-enylmethyl-piperidin-4-yl)acetamide;
2-benzofuran-4-yl-N-[1-(6,6-dimethyl-bicyclo[3.1.1]hept-2-en-2-ylmethyl)piperidin-4-yl]acetamide;
2-benzofuran-4-yl-N-[1-(2-ethyl-hex-2-enyl)piperidin-4-yl]acetamide;
2-benzofuran-4-yl-N-[1-(3,5,5-trimethylhexyl)piperidin-4-yl]acetamide;
2-benzofuran-4-yl-N-[1-((S)-6,6-dimethyl-bicyclo[3.1.1]hept-2-ylmethyl)piperidin-4-yl]acetamide;
2-benzofuran-4-yl-N-[1-(2-cyclohexylpropyl)piperidin-4-yl]acetamide;
2-benzofuran-4-yl-N-[1-(5-fluoro-2,3-dihydrobenzofuran-2-ylmethyl)piperidin-4-yl]acetamide;
2-benzofuran-4-yl-N-[1-(2-phenoxyethyl)piperidin-4-yl]acetamide;
2-benzofuran-4-yl-N-[1-(3-phenylpropyl)piperidin-3-yl]acetamide;
2-benzofuran-4-yl-N-(1-benzylpiperidin-4-yl)acetamide;
3-benzofuran-2-yl-N-[1-(3,4-dichlorobenzyl)piperidin-4-yl]propionamide;
2-benzofuran-4-yl-N-[1-(3-phenylpropylpiperidin-4-yl]acetamide;
2-benzofuran-4-yl-N-[3,4-dichlorobenzyl)piperidin-4-yl]acetamide, formate salt;
2-benzofuran-4-yl-N-{1-[3-(3,4-dichloro-phenyl)-propyl]-piperidin-4-yl}-acetamide;
2-benzofuran-4-yl-N-[1-(2-methyl-3-phenyl-propyl)-piperidin-4-yl] acetamide;
and the salts, solvates, hydrates and N-oxides thereof.

16. A pharmaceutical composition comprising a compound according,to claim 1, together with one or more pharmaceutically acceptable carriers, excipients or diluents.

17. Use of a compound of claim 1, for the manufacture of a medicament for the treatment of immune or inflammatory disorders.
